(19) United States
(12) Patent Application Publication
(10) Pub. No.: US 2024/0108617 A1
(43) Pub. Date: Apr. 4, 2024

(54) AN INHALABLE DRY POWDER COMPOSITION FOR PULMONARY DISEASES

(71) Applicant: GLENMARK SPECIALTY S.A., La Chaux-De-Fonds (CH)

(72) Inventors: Sushrut KULKARNI, Navi Mumbai (IN); Rajesh Verbeerabhadrarao ANKAM, Nashik (IN); Rakshit Kanubhai TRIVEDI, Nashik (IN); Vaijnath Shadakshari ARAVAT, Nashik (IN); Vijay Ganpat LUGADE, Nashik (IN); Someshwar Dashrath NAVHAT, Nashik (IN)

(21) Appl. No.: 17/754,678

(22) PCT Filed: Oct. 11, 2020
(86) PCT No.: PCT/IB2020/059541

§ 371 Date: Apr. 8, 2022

(30) Foreign Application Priority Data
Oct. 11, 2019 (IN) 201921041251

(51) Int. Cl. A61K 31/4965; A61K 9/00; A61K 9/16; A61K 9/48; A61K 47/26

(52) U.S. Cl. CPC A61K 31/4965; A61K 9/0075; A61K 9/16; A61K 9/1682; A61K 9/4833; A61K 47/26

(57) ABSTRACT

The present invention relates to an inhalable composition comprising a therapeutically effective dosage amount of compound (I), 2-{4-[(2S)-1,1-difluoro-2-hydroxypropyl]phenyl}-N-{4-[3-(2-ethylphenyl) pyrazine-2-yl]phenyl}acetamide or a pharmaceutically acceptable salt thereof. Particularly, the present invention relates to a inhalable dry powder composition comprising compound (I) or a pharmaceutically acceptable salt thereof. Further, the present invention relates to a method of treating a pulmonary disease using such a composition.

### FIELD OF THE INVENTION

The present invention relates to an inhalable dry powder composition comprising a therapeutically effective dosage amount of compound (I), 2-{4-[(2S)-1,1-difluoro-2-hydroxypropyl]phenyl}-N-{4-[3-(2-ethylphenyl) pyrazine-2-yl]phenyl}acetamide or a pharmaceutically acceptable salt thereof. Further, the present invention relates to a method of treating a pulmonary disease using such a composition.

### BACKGROUND OF THE INVENTION

Respiratory diseases related to airway inflammation include a number of lung diseases such as symptoms of pulmonary infection(s), caused by bacteria, virus, or some types of fungus such as mild to moderate breathing difficulty, chronic obstructive pulmonary disease (COPD) and asthma.

Asthma is a disease characterized by an increased responsiveness of the trachea and bronchi to various stimuli, and manifested by widespread narrowing of the airways that changes in severity either spontaneously or as a result of treatment. The events leading to airway obstruction in asthma include edema of airway walls, infiltration of inflammatory cells into the lung, production of various inflammatory mediators and increased mucous production.

The current therapy for asthma includes bronchodilator drugs, corticosteroids and leukotriene antagonists. Bronchodilator drugs dilate the bronchi and bronchioles, decreasing resistance in the respiratory airway and increasing airflow to the lungs. Corticosteroid drugs are effective at reducing asthma symptoms by blocking the body's inflammatory response. The leukotriene antagonists have limited efficacy, with only small increases in pulmonary function demonstrated in clinical trials.

COPD is a term used to classify two major airflow obstruction diseases: chronic bronchitis and emphysema. Chronic bronchitis is inflammation of the bronchial airways. Emphysema is inflammation of the alveoli, or air sacs in the lungs. Emphysema has a number of causes, including smoking, exposure to environmental pollutants, alpha-one antitrypsin deficiency, and aging. COPD is a disease of the respiratory apparatus, characterized by an irreversible obstruction of the airways, of a degree that varies according to the gravity.

Compound (I) is chemically known as 2-{4-[(2S)-1,1-difluoro-2-hydroxypropyl]phenyl}-N-{4-[3-(2-ethylphenyl) pyrazine-2-yl]phenyl}acetamide or a pharmaceutically acceptable salt thereof. Compound (I) is an orally active, potent and selective RORγ modulator, disclosed in WO 2017/021879 (the '879 application), incorporated herein by reference in its entirety, for the treatment of a disease or condition modulated by RORγ such as asthma, COPD, rheumatoid arthritis, and other inflammatory diseases. The '879 application broadly discloses a pharmaceutical composition for compound (I).

Pulmonary delivery of therapeutic agents can offer several advantages over other modes of delivery. These advantages include rapid onset, the convenience of patient self-administration, the potential for reduced drug side-effects, ease of delivery by inhalation, the elimination of needles, and the like. Inhalation therapy is capable of providing a drug delivery system that is easy to use in an inpatient or outpatient setting, results in very rapid onset of drug action, and produces minimal side effects. There are variety of dosage forms meant for pulmonary administration such as metered dose inhalers, liquid aerosol delivery, nebulizers, dry powder inhalers and the like. The applicant has now found that inhalation compositions are suitable dosage form for compound (I), specifically dry powder inhaler composition.

The applicant has now found that inhalable composition comprising compound (I) and pharmaceutically acceptable salts thereof that is suitable for pulmonary administration.

### SUMMARY OF THE INVENTION

The present invention relates to an pharmaceutical composition comprising compound (I) or a pharmaceutically acceptable salt thereof.

One embodiment is an inhalable composition comprising compound (I) or a pharmaceutically acceptable salt thereof.

Another embodiment is an inhalable composition comprising a therapeutically effective amount of compound (I) or a pharmaceutically acceptable salt thereof and one or more pharmaceutically acceptable excipient(s).

Another embodiment is an inhalable dry powder composition comprising compound (I) or a pharmaceutically acceptable salt thereof.

Another embodiment is an inhalable dry powder composition comprising a therapeutically effective dosage amount of compound (I) or a pharmaceutically acceptable salt thereof.

Yet another embodiment is an inhalable dry powder composition comprising a therapeutically effective dosage amount of compound (I) or a pharmaceutically acceptable salt thereof for preventing, inhibiting, suppressing, and/or treating at least one pulmonary disease.

In some embodiments, the pulmonary disease is selected from asthma, COPD, bronchitis, chronic or acute bronchoconstriction, adult respiratory distress syndrome, acute lung injury, and bronchiectasis.

Yet another embodiment is an inhalable dry powder composition comprising a therapeutically effective dosage amount of compound (I) or a pharmaceutically acceptable salt thereof for preventing, inhibiting, suppressing, and/or treating COPD.

The inhalable dry powder composition may comprise a therapeutically effective amount of compound (I) or a pharmaceutically acceptable salt thereof and one or more pharmaceutically acceptable excipient(s).

Another embodiment is a composition comprising a therapeutically effective dosage amount of compound (I) or a pharmaceutically acceptable salt thereof.

In one embodiment, the composition is a capsule dosage form.

One embodiment is an inhalable dry powder composition comprising a therapeutically effective dosage amount of compound (I) or a pharmaceutically acceptable salt thereof, in the range of from about 0.1 mg to about 20 mg and one or more pharmaceutically acceptable excipient(s).

Yet another embodiment is an inhalable dry powder composition comprising a therapeutically effective dosage amount of compound (I) or a pharmaceutically acceptable salt thereof, in the range of from about 0.1 mg to about 20 mg and a diluent.

Yet another embodiment is an inhalable dry powder composition comprising: a therapeutically effective dosage amount of compound (I) or a pharmaceutically acceptable salt thereof, in the range of from about 0.1 mg to about 20 mg and a lactose component.

Yet another embodiment is an inhalable dry powder composition comprising a therapeutically effective dosage amount of compound (I) or a pharmaceutically acceptable salt thereof, in the range of from about 0.1 mg to about 20 mg and a lactose component having a d₉₀ particle size less than about 60 microns.

Yet another embodiment is an inhalable dry powder composition comprising a therapeutically effective dosage amount of compound (I) or a pharmaceutically acceptable salt thereof, in the range of from about 0.1 mg to about 20 mg and a lactose component having a d₉₀ particle size less than about 200 microns.

Yet another embodiment is an inhalable dry powder composition comprising a therapeutically effective dosage amount of compound (I) or a pharmaceutically acceptable salt thereof, in the range of from about 0.1 mg to about 20 mg and a lactose component, wherein the lactose component comprises i) lactose particles having a d₉₀ particle size less than about 60 microns and ii) lactose particles having a d₉₀ particle size less than about 200 microns.

Yet another embodiment is an inhalable dry powder composition comprising a therapeutically effective dosage amount of compound (I) or a pharmaceutically acceptable salt thereof, in the range of from about 0.1 mg to about 20 mg and a lactose component, wherein the lactose component has an average particle size of from about 1 microns to about 200 microns.

In some embodiments, the inhalable dry powder composition is physically and chemically stable.

In an embodiment, the present invention relates to a method of preventing, inhibiting, suppressing, and/or treating at least one pulmonary disease(s) characterized by airflow obstruction or limitation, or a symptom occurring in pulmonary region, including but not limited to asthma, emphysema, bronchitis, COPD, sinusitis, respiratory depression, reactive airways dysfunction syndrome (RADS), acute respiratory distress syndrome (ARDS), irritant induced asthma, occupational asthma, symptoms of pulmonary infection(s), sensory hyper-reactivity, airway (or pulmonary) inflammation, multiple chemical sensitivity, aid in smoking cessation therapy, pneumonia, bronchitis, influenza, SARS, SARS-CoV-2 (COVID-19), tuberculosis, and whooping cough (pertussis), and the like in a subject in need thereof.

Yet another embodiment is a method of preventing, inhibiting, suppressing, and/or treating at least one pulmonary disease in a subject in need thereof comprising administering a therapeutically dosage effective amount of compound (I) or a pharmaceutically acceptable salt thereof to the subject via inhalation.

Yet another embodiment is a method of treating symptom(s) of pulmonary infection(s) in a subject in the need thereof, comprising administering a therapeutically effective dosage amount of compound (I) or a pharmaceutically acceptable salt thereof to the subject via inhalation.

In some embodiments, the pulmonary infection(s) includes viral, bacterial, or fungal infection. The symptoms of pulmonary infection(s) is mild to severe breathing problem, dry or productive cough, chest pain, whooping cough and/or sore throat. Yet another embodiment is a method of preventing, inhibiting, suppressing, and/or treating at least one pulmonary disease in a subject in need thereof comprising administering an inhalable dry powder composition comprising compound (I) or a pharmaceutically acceptable salt thereof to the subject via inhalation.

Yet another embodiment is a method of preventing, inhibiting, suppressing, and/or treating at least one pulmonary disease in a subject in need thereof comprising administering an inhalable dry powder composition comprising a therapeutically effective dosage amount of compound (I) or a pharmaceutically acceptable salt thereof to the subject via inhalation.

In one embodiment, the pulmonary disease is one or more condition(s) selected from asthma, emphysema, bronchitis, COPD, sinusitis, respiratory depression, reactive airways dysfunction syndrome (RADS), acute respiratory distress syndrome (ARDS), irritant induced asthma, occupational asthma, symptoms of pulmonary infection(s), sensory hyper-reactivity, airway (or pulmonary) inflammation, multiple chemical sensitivity, aid in smoking cessation therapy, pneumonia, bronchitis, influenza, SARS, SARS-CoV-2 (COVID-19), tuberculosis, and whooping cough (pertussis).

Yet another embodiment is a method of preventing, inhibiting, suppressing, and/or treating COPD comprising administering a therapeutically effective dosage amount of compound (I) or a pharmaceutically acceptable salt thereof to the subject via inhalation.

Yet another embodiment is a method of preventing, inhibiting, suppressing, and/or treating COPD comprising administering an inhalation powder composition comprising a therapeutically effective dosage amount of compound (I) or a pharmaceutically acceptable salt thereof to the subject via inhalation.

Yet another embodiment is a method of administering, comprising administering compound (I) or a pharmaceutically acceptable salt thereof to the subject via inhalation.

In one embodiment, the method of administration comprises administering a therapeutically effective amount of compound (I) or a pharmaceutically acceptable salt thereof with an inhalation device.

Yet another embodiment is a method of administering inhalable dry powder composition comprising a therapeutically effective dosage amount of compound (I) or a pharmaceutically acceptable salt thereof to the subject; wherein the method comprises administration of a unit dosage rapidly with one or two, small volume breaths as needed.

Yet another embodiment is a process for preparing an inhalable dry composition comprising compound (I) or a pharmaceutically acceptable salt thereof comprising: i) mixing a therapeutically effective dosage amount of compound (I) or a pharmaceutically acceptable salt thereof with one or more pharmaceutically acceptable excipient(s), and ii) filling the blend in a capsule/blister/sachet dosage form meant for unit dose dry powder inhaler.

### DETAILED DESCRIPTION

The terms used herein are defined as follows. If a definition set forth in the present application and a definition set forth later in a non-provisional application claiming priority from the present provisional application are in conflict, the definition in the non-provisional application shall control the meaning of the terms.

The term singular forms “a,” “an” and “the” include plural references unless the context clearly dictates otherwise. Thus, for example, reference to “an excipient” includes a single excipient as well as two or more different excipients, and the like.

The chemical compound referred herein as compound (I) in the context of the present invention has the following chemical structure:

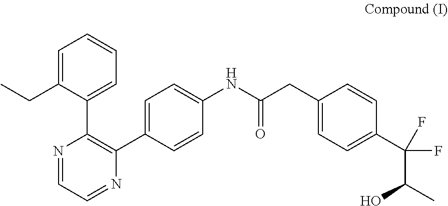 [SMILES: CCc1ccccc1-c1nccnc1-c1ccc(NC(=O)Cc2ccc(C(F)(F)[C@@H](C)O)cc2)cc1]

Compound (I) is chemically known as 2-(4-(1,1-difluoro-2-hydroxypropyl)phenyl)-N-(4-(3-(2-ethylphenyl)pyrazin-2-yl)phenyl)acetamide. In a preferred embodiment, compound (I) is 2-{4-[(2S)-1,1-difluoro-2-hydroxypropyl]phenyl}-N-{4-[3-(2-ethylphenyl) pyrazine-2-yl] phenyl}acetamide.

In another embodiment, the compound (I) may exist in amorphous or any crystalline form.

In a specific embodiment, the compound (I) may exist in crystalline Form III and is characterized by the characteristic X-ray diffraction pattern comprising one or more of the following peaks expressed in terms of 11.3, 12.1, 15.9, 18.1 and 20.0±0.2° theta.

In a specific embodiment, the compound (I) may exist as racemic mixture of S and R isomers.

In a specific embodiment, the compound (I) may exist as R-isomer.

In a specific embodiment, the compound (I) may exist as S-isomer.

The term “pharmaceutically acceptable salt” as used herein refers to salt forms which are within the scope of sound medical judgment, suitable for use in contact with the tissues of humans and lower animals without undue toxicity, irritation, and allergic response, commensurate with a reasonable benefit to risk ratio, and effective for their intended use.

The term “pharmaceutically acceptable salt” of compound (I) includes salts prepared from pharmaceutically acceptable bases or acids including inorganic or organic bases and inorganic or organic acids. Examples of such salts include, but are not limited to, acetate, benzenesulfonate, benzoate, bicarbonate, bisulfate, bitartrate, borate, bromide, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, N-methylglucamine ammonium salt, oleate, oxalate, pamoate (embonate), palmitate, pantothenate, phosphate, diphosphate, polygalacturonate, salicylate, stearate, sulfate, subacetate, succinate, tannate, tartrate, teoclate, tosylate, triethiodide and valerate. Examples of salts derived from inorganic bases include, but are not limited to, aluminum, ammonium, calcium, copper, ferric, ferrous, lithium, magnesium, manganic, mangamous, potassium, sodium, and zinc.

In one preferred embodiment, compound (I) is in its free base form.

The term “treating” as used herein refers to reduction in severity and/or frequency of symptoms, amelioration suppression, elimination of symptoms and/or their underlying cause, or any clinical improvement of a disease or condition.

The term “prevention” as used herein refers to prophylaxis from a disease or condition or its symptoms. In some embodiments, the term “prevention” refers to preventing relapse/exacerbation of pulmonary diseases and/or symptoms.

As used herein, the term “about” when used to refer to weight % in a composition or other numeral amounts means plus or minus up to 20% (alternatively, up to 10% or 5%) of the reported value.

The term “subject” includes mammals such as humans and domestic animals (e.g., household pets including cats and dogs) and non-domestic animals (such as wildlife). Preferably, the subject is a human.

The term “therapeutically effective amount” as used herein refers to an amount of an active agent that is sufficient to provide the desired therapeutic effect. The amount required for exhibiting therapeutic activity in the composition, and the therapeutic effective amount is in the range of from about 0.1 nM to about 20 μM.

The term “therapeutically effective dosage amount” as used herein refers to an amount of the active agent that is incorporated in the dosage form to deliver a therapeutically effective amount at the target site. For example, the therapeutically effective dosage amount of compound (I) or a pharmaceutically acceptable salt thereof may be selected from about 0.1 mg to about 20 mg. Examples of doses include about 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, 10, 10.1, 10.2, 10.3, 10.5, 10.6, 10.7, 10.8, 10.9, 11, 11.1, 11.2, 11.3, 11.4, 11.5, 11.6, 11.7, 11.8, 11.9, 12, 12.1, 12.2, 12.3, 12.4, 12.5, 12.6, 12.7, 12.8, 12.9, 13, 13.1, 13.2, 13.3, 13.4, 13.5, 13.6, 13.7, 13.8, 13.9, 14, 14.1, 14.2, 14.3, 14.4, 14.5, 14.6, 14.7, 14.8, 14.9, 15, 15.1, 15.2, 15.3, 15.4, 15.5, 15.6, 15.7, 15.8, 15.9, 16, 16.1, 16.2, 16.3, 16.4, 16.5, 16.6, 16.7, 16.8, 16.9, 17, 17.1, 17.2, 17.3, 17.4, 17.5, 17.6, 17.7, 17.8, 17.9, 18, 18.1, 18.2, 18.3, 18.4, 18.5, 18.6, 18.7, 18.8, 18.9, 19, 19.1, 19.2, 19.3, 19.4, 19.5, 19.6, 19.7, 19.8, 19.9, and 20 mg.

In a specific embodiment, the therapeutically effective dosage amount is selected from about 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, 10, 10.1, 10.2, 10.3, 10.5, 10.6, 10.7, 10.8, 10.9, 11, 11.1, 11.2, 11.3, 11.4, 11.5, 11.6, 11.7, 11.8, 11.9, and 12 mg.

The term “pharmaceutically acceptable excipients” as used herein refers to any of the components of a pharmaceutical composition other than the active agents and which are approved by regulatory authorities or are generally regarded as safe for human or animal use.

The term “unit dose” or “unit dosage form” as used herein refers to inhalable dry powder composition filled in a unit dose container. The unit dose container herein refers to a capsule (e.g. size 000, 00, 0E, 0, 1, 2, 3, and 4, with respective volumetric capacities of 1.37 ml, 950 μl, 770 μl, 680 μl, 480 μl, 360 μl, 270 μl, and 200 μl) or a blister or a sachet.

As used herein, the term “average particle size” (or synonymously, “mean particle size”) refers to the distribution of particles, wherein about 50 volume percent of all the particles measured have a size less than the defined average particle size value and about 50 volume percent of all measurable particles measured have a particle size greater than the defined average particle size value. This can be identified by the term “d₅₀” or “d (0.5)”. Similarly, d₉₀ value relates to about 90 volume percent of all the particles measured have a size less than the defined particle size value (also referred to as “d₉₀ particle size’). The particle size can be measured using various techniques like laser diffraction, photon correlation spectroscopy (PCS) and Coulter's principle.

The term “d₉₀ particle size” as used herein refers to the diameter of particles where ninety percent of the distribution has a smaller particle size and ten percent has a larger particle size.

The “fine particle mass” test is normally conducted using a validated multistage impactor or impinger method, or a suitably validated alternative. It is normally considered acceptable to set upper and lower limits on the results of pooled stages corresponding to an average particle size of less than 5 microns, although alternative limits may be found acceptable with adequate justification. The drug mass should be reported in fine particle mass rather than the percentage of emitted dose (or other derived parameter). The fine particle mass is generally mentioned as mass/dose. For example, if the inhalable dry powder composition has fine particle mass of 50 mcg, means fine particle mass is 50 mcg/dose.

The term “aerodynamic diameter” is defined as the diameter of a sphere of density 1000 kg/m³ with the same settling velocity as the particle of interest. Aerodynamic diameters may be ascertained by any of the methods customarily used by those in the art. Aerodynamic diameter values specified herein are as determined using Anderson Cascade Impactor.

The Mass Median Aerodynamic Diameter (MMAD) is defined as the diameter at which 50% of the particles by mass are larger and 50% are smaller.

The “Carr index” and “Hausner ratio” are frequently used in as an indication of the flowability of a powder. The Carr index and Hausner ratio can be determined using Tap Density Apparatus. The bulk density and tap density of the powder composition can be determined by Tap density apparatus.

The term “fine particle fraction” is defined as a respirable fraction of emitted particles, meaning that are less than a particle size that is considered the upper limit respirable. The fine particle fraction has aerodynamic diameter less than 5 microns. The fine particle fraction generally from about 5 microns to about 1 micron MMAD.

The term “pulmonary disease” as used herein refers to a disease, a disorder or a condition, or a symptom that occurs in the pulmonary system. Pulmonary diseases include, but are not limited to, asthma, emphysema, bronchitis, COPD, sinusitis, respiratory depression, reactive airways dysfunction syndrome (RADS), acute respiratory distress syndrome (ARDS), irritant induced asthma, occupational asthma, sensory hyper-reactivity, airway (or pulmonary) inflammation, multiple chemical sensitivity, aid in smoking cessation therapy, chronic or acute bronchoconstriction, acute lung injury, bronchiectasis, pneumonia, bronchitis, influenza, SARS, SARS-CoV-2 (COVID-19), tuberculosis, and whooping cough (pertussis).

The term “lactose” or “lactose component” as used herein, without any limitation, refers to any inhalable grade lactose or surface-modified lactose or lactose prepared by a process in which specific surface area of the lactose is about 0.15 m2/g to about 0.3 m2/g

In the context of present invention, the inhalation composition comprises intranasal or oral inhalation or both, particularly wherein the compound (I) is administered to the lungs or pulmonary region of the subject.

The present invention also provides a pharmaceutical composition for inhalation administration comprising compound (I) or a pharmaceutically acceptable salt thereof. Pharmaceutical compositions suitable for administration by the inhalation route include oral and nasal inhalation formulations such as dry powder inhaler (DPI) formulations, metered dose inhaler (MDI) formulations (including oral and nasal aerosols), nasal sprays, and formulations suitable for nebulization.

The present invention relates to an inhalable composition comprising compound (I) or a pharmaceutically acceptable salt thereof.

In another embodiment, the inhalable composition of the present invention comprises compound (I) or a pharmaceutically acceptable salt thereof and one or more pharmaceutically acceptable excipient(s).

In another embodiment, the inhalable composition of the present invention comprises a therapeutically effective dose of compound (I) or a pharmaceutically acceptable salt thereof and one or more pharmaceutically acceptable excipient(s).

In a specific embodiment, the inhalation composition is nasal spray.

In a specific embodiment, the inhalation composition that is suitable for nebulization.

In a specific embodiment, the inhalation composition is metered dose inhalation composition (MDI). The metered dose inhalation composition alternatively means aerosol composition with propellant.

In some embodiments, the inhalation composition of the present invention relates to an inhalable composition comprising compound (I) or a pharmaceutically acceptable salt thereof and one or more pharmaceutically acceptable excipient(s) thereof.

The compound (I) can be in the form of solution or suspension in the composition.

In some embodiments the present invention relates to an inhalable composition comprising an therapeutically effective dosage amount of compound (I) or a pharmaceutically acceptable salt and one or more pharmaceutically acceptable excipient(s) to be delivered by pressurized metered dose inhalers suitable for pulmonary administration.

The inhalable composition is aerosol composition delivered via pressurized metered dose inhalers, alternatively called as MDI. The MDI composition additionally comprises one or more propellants such as HFA propellant. A suitable HFA propellant is toxicologically safe and must have a vapor pressure in order to enable the medicament to be administered via a pressurized MDI. An HFA propellant can be selected from HFA-134(a), HFA-227A, HFA-32 HFC-143(a), HFC-134, HFC-152a, and mixture thereof, preferably, HFA-134(a) and HFA-227. More preferably, the HFA propellant is HFA-134(a). The MDI compositions of the present invention may additionally comprise one or more solvents and/or co-solvents selected from one or more of C₂-C₆ aliphatic alcohols (such as, but not limited to, ethyl alcohol and isopropyl alcohol), glycerol, polyoxyethylene alcohols, polyoxyethylene fatty acid esters, hydrocarbons (such as, but not limited to, n-propane, n-butane, isobutane, n-pentane, iso-pentane, neo-pentane, and n-hexane), and ethers (such as but not limited to diethyl ether). The alcoholic co-solvent in the present invention comprises one or more of C₂-C₆ aliphatic alcohols, glycerol, polyoxyethylene alcohols, wherein co-solvent may further comprise water. More preferably, the co-solvent is anhydrous ethanol. The MDI composition of the present invention may additionally comprises one or more stabilizer(s) selected from one or more organic acid selected from, but not limited to, citric acid, tartaric acid, lactic acid, oleic acid, formic acid, acetic acid, oxalic acid, ascorbic acid, malic acid and succinic acid or mixtures thereof; and one or more ionic and/or non-ionic surfactants including, but not limited to, salts of stearic acids such as magnesium stearate, esters such as ascorbyl palmitate, isopropyl myristate and tocopherol esters, lecithin, tyloxapol, polysorbates such as polysorbate 80, polysorbate 20, and polysorbate 40, vitamin E-TPGS, macrogol hydroxystearates such as macrogol-15-hydroxystearate, acetylated monoglycerides such as Myvacet 9-45 and Myvacet 9-08, polyoxyethylene ethers, ethyloleate, glyceryl trioleate, glyceryl monolaurate, glyceryl monooleate, glyceryl monosterate, glyceryl monoricinoleate, cetylalcohol, sterylalcohol, cetylpyridinium chloride, block polymers, natural oils, polyvinyl pyrrolidone, sorbitan fatty acid esters such as sorbitan trioleate, polyethoxylated sorbitan fatty acid esters (for example polyethoxylated sorbitan trioleate), sorbimacrogol oleate, synthetic amphotensides (tritons), ethylene oxide ethers of octylphenolformaldehyde condensation products.

In a more specific embodiment, the inhalation composition is dry powder composition.

The present invention preferrably relates to an inhalable dry powder composition comparing a therapeutically effective dosage amount of compound (I) or a pharmaceutically acceptable salt thereof.

In an embodiment, the present invention relates to an inhalable dry powder composition comprising: a therapeutically effective dosage amount of compound (I) or a pharmaceutically acceptable salt thereof, and one or more pharmaceutically acceptable excipient(s) thereof.

The dry powder composition of the present invention can comprise further components which is commonly used in the preparation of a dry powder composition that is to be administered using a dry powder inhaler. Thus, the dry powder composition may further comprise one or more pharmaceutically acceptable excipient(s). The pharmaceutically acceptable excipient(s) function as a diluent particles. The pharmaceutically acceptable excipient provide bulk, which improves handling, dispensing, and metering of the compound (I) of the present invention. An example of such pharmaceutically acceptable excipient is lactose, or mannitol or glucose. The pharmaceutically acceptable excipient(s) of composition of the present invention makes at least 99.5% to about 30% based on the total weight of the composition. Despite lack of choice of pharmaceutically acceptable excipient(s) in dry powder composition, the excipient must be chosen carefully by considering various physiochemical character of the excipient such as particle size, morphology profoundly affect the performance of the composition. The inadequate separation of compound (I) and pharmaceutically acceptable excipient can lead to lack of content uniformity and may provide deposition problems. It is also to be considered that pharmaceutically acceptable excipient(s) or diluent(s) for dry powder composition is not always required, and sometime, dry powder composition may comprise only compound (I) without pharmaceutically acceptable excipient(s).

The inhalable dry powder composition in the above embodiments may optionally comprise one or more pharmaceutically acceptable excipients.

The inhalable dry powder composition may contain one or more pharmaceutically acceptable excipients. Examples of such excipients include, but are not limited to, diluents and solvents.

Suitable pharmaceutically acceptable excipient(s) include, but are not limited to, lactose, surface-modified lactose, glucose, maltose, erythritol, sorbitol, raffinose, xylitol, maltitol, inositol, fructose, sucrose, dextrose, mannitol, trehalose, cyclodextrin their hydrates and mixtures thereof. Their average particle sizes of these excipient(s) may be the same or different. It should be noted that the dry powder composition may comprise one or more excipients selected from lactose, glucose, maltose, erythritol, sorbitol, raffinose, xylitol, maltitol, inositol, fructose, sucrose, dextrose, mannitol, trehalose, and cyclodextrin or any combinations of any of the foregoing.

The pharmaceutically acceptable diluent suitable for use in the invention is selected from lactose, surface-modified lactose, glucose, maltose, erythritol, sorbitol, raffinose, xylitol, maltitol, inositol, fructose, sucrose, dextrose, mannitol, trehalose, cyclodextrin their hydrates and mixtures thereof.

Preferably, the pharmaceutically acceptable diluent is lactose. Various inhalable grades of lactose are available for use in dry powder compositions are selected from Respitose® SV010, Respitose® SV003, Respitose® ML006, Lactose Monohydrate Inhalation 40M, Lactose Anhydrous 120M, Lactohale® 300, Lactohale® 230, Lactohale® 220, Lactohale® 210, Lactohale® ML001, Lactohale® 206, Lactohale® 200, Lactohale® 800, Lactohale® 100 and the like.

In some embodiments, the pharmaceutically acceptable diluent is surface-modified lactose. The surface of lactose particle are modified, i.e., for example, particle soothing is done, in order to produce high dispersible lactose particle to use in inhalable dry powder composition.

In some embodiments, the pharmaceutically acceptable diluent is lactose in which the lactose is prepared by a process comprising sifting the unprocessed lactose through a sieve of suitable mesh size until desired reduction in weight is obtained. In one embodiment the suitable mesh size is selected from about 20 microns to about 60 microns. More preferably, the suitable mesh size is selected from about 30 microns to 50 microns. In a still preferred embodiment, the suitable mesh size is 38 microns.

The term “Respitose ML006” as used herein refers to inhalable grade lactose monohydrate, available from DFE Pharma of Goch, Germany, is a fine milled inhalation grade lactose with a narrow particle size distribution.

The term “Respitose SV010” as used herein refers to an inhalable grade lactose monohydrate, available from DFE Pharma of Goch, Germany, with a relatively broad particle size distribution.

The term “Inhalac70” as used herein refers to an inhalable grade lactose monohydrate, available from MEGGLE Excipients & Technologie, Germany.

The term “Lactohale LH200” as used herein to refer an inhalable grade lactose monohydrate, available from DFE Pharma of Goch, Germany, is a coarser version of lactose (particle size 120-160 microns).

The term “Lactohale LH300” as used herein to refer an inhalable grade lactose monohydrate available from DFE Pharma of Goch, Germany, is a finer version of lactose (particle size less than 10 microns).

The dry powder composition of the present invention may further comprise a lubricant. A lubricant can be, but not limited to, one or more excipient selected from leucine, magnesium stearate, sodium stearate or polyethylene glycol. It is also possible to use more than one lubricant in the dry powder composition of the present invention.

The powder composition may be further filled into a capsule for inhalation or may be processed into a lightly compressed tablet or powder agglomeration which can be easily crushed to obtain a powder for inhalation. Alternately, the composition can be filled, either as discrete dosage units, in a capsule, blister or a sachet. The capsule can be a hard gelatin capsule or gelatin free hard capsule.

In a specific embodiment, the pharmaceutically acceptable excipient is a lactose component. The lactose component may be one or more lactose(s) having a d90 particle size from about 1 microns to about 200 microns.

In a specific embodiment, the lactose component may be one or more lactose(s) having a d90 particle size from about 2 microns to 185 microns.

In an embodiment, the inhalable dry powder composition of the present invention is in a unit dosage form.

In additional embodiments, the inhalable dry powder composition of the present invention is filled in a capsule/blister/sachet dosage form meant for a monodose/a unit dose i.e., an inhalable dry powder composition in a capsule/blister/sachet meant for unit dose dry powder inhaler and can be delivered to the user from the container by suction.

One embodiment is an inhalable dry powder composition comprising a therapeutically effective dosage amount of compound (I) or a pharmaceutically acceptable salt thereof, and a pharmaceutically acceptable excipient(s) thereof.

In yet another embodiment is an inhalable dry powder composition comprising a therapeutically effective dosage amount of compound (I) or a pharmaceutically acceptable salt thereof, in the range of from about 0.1 mg to about 20 mg and one or more pharmaceutically acceptable excipient(s).

In some embodiments, the compound (I) or a pharmaceutically acceptable salt thereof is micronized.

In some embodiments, the compound (I) or a pharmaceutically acceptable salt thereof is unmicronized.

In some embodiments, the compound (I) or a pharmaceutically acceptable salt thereof is mixture of micronized and unmicronized particles.

The average particle size of compound (I) or a pharmaceutically acceptable salt thereof is from about 0.1 microns to about 10 microns.

In a specific embodiment, the d₉₀ particle size of compound (I) or a pharmaceutically acceptable salt thereof is less than about 10 microns.

In a specific embodiment, the d₉₀ particle size of compound (I) or a pharmaceutically acceptable salt thereof is less than about 8 microns or less than about 7 microns or less than about 6 microns or less than about 5 microns or about 5 microns or about 4 microns or about 3 microns or about 2 microns.

In a specific embodiment, the d₉₀ particle size of the compound (I) or a pharmaceutically acceptable salt thereof is not more than about 5 microns.

In a specific embodiment, the d₁₀ particle size of compound (I) or a pharmaceutically acceptable salt thereof is more than about 0.1 microns.

In a specific embodiment, the d₅₀ particle size of compound (I) or a pharmaceutically acceptable salt thereof is in the range of from about 0.7 microns to about 4 microns.

In a specific embodiment, the aerodynamic particle size of the compound (I) or a pharmaceutically acceptable salt thereof is in the range of from about 0.1 microns to about 10 microns.

In a specific embodiment, the aerodynamic particle size of the compound (I) or a pharmaceutically acceptable salt thereof is less than about 10 microns.

In a specific embodiment, the aerodynamic particle size of the compound (I) or a pharmaceutically acceptable salt thereof is less than about 9 microns.

In a specific embodiment, the aerodynamic particle size of the compound (I) or a pharmaceutically acceptable salt thereof is less than about 8 microns.

In a specific embodiment, the aerodynamic particle size of the compound (I) or a pharmaceutically acceptable salt thereof is less than about 7 microns.

In a specific embodiment, the aerodynamic particle size of the compound (I) or a pharmaceutically acceptable salt thereof is less than about 6 microns.

In a specific embodiment, the aerodynamic particle size of the compound (I) or a pharmaceutically acceptable salt thereof is less than about 5 microns.

In a specific embodiment, the aerodynamic particle size of the compound (I) or a pharmaceutically acceptable salt thereof is in the range of from about 0.1 microns to about 5 microns.

The particle size of compound (I) has direct influence on rate of absorption and in turn bioavailability of insoluble compound such as compound (I), hence, particle size of compound (I) is optimized to less than about 5 microns.

In some embodiments, the particles of compound (I) may be coated with one or more polymeric excipient(s). A polymer coating can include, but is not limited to any one of the following, chitosan, polyvinyl alcohol, polylactic-co-glycolic acid, or sugar polymers (e.g. dextran, dextrin). It is also possible to use more than one kind of polymer coating in the dry powder composition of the present invention.

One embodiment is an inhalable dry powder composition comprising i) a therapeutically effective dosage amount of compound (I) or a pharmaceutically acceptable salt thereof; and one or more pharmaceutically acceptable excipient(s), wherein the average particle size of compound (I) or a pharmaceutically acceptable salt thereof is from about 0.1 microns to about 10 microns.

In another embodiment is an inhalable dry powder composition comprising i) a therapeutically effective dosage amount of compound (I) or a pharmaceutically acceptable salt thereof; and one or more pharmaceutically acceptable excipient(s), wherein the aerodynamic particle size of compound (I) or a pharmaceutically acceptable salt thereof is from about 0.1 microns to about 10 microns.

In a preferred embodiment is an inhalable dry powder composition comprising i) a therapeutically effective dosage amount of compound (I) or a pharmaceutically acceptable salt thereof; and one or more pharmaceutically acceptable excipient(s), wherein the aerodynamic particle size of compound (I) or a pharmaceutically acceptable salt thereof is from about 0.1 microns to about 5 microns.

In a preferred embodiment, the pharmaceutically acceptable excipient is a lactose component, and the lactose component is present in an amount more than about 30%, based upon 100% total weight of the composition.

One embodiment is an inhalable dry powder composition comprising a therapeutically effective dosage amount of compound (I) or a pharmaceutically acceptable salt thereof and a lactose component.

The lactose component may comprise one or more inhalation grade lactose particles, each having a different average particle size.

The lactose component may comprise one or more inhalation grade lactose particles each having different aerodynamic diameter.

In one embodiment, the lactose component is lactose monohydrate having an average particle size of from about 1 microns to about 200 microns.

In one embodiment, the lactose component is selected from (i) lactose particles having a d₉₀ of from about 1 microns to about 60 microns, (ii) lactose particles having a d₉₀ of from about 50 microns to about 200 microns, and (iii) mixtures thereof.

In one embodiment, the lactose component is selected from (i) lactose particles having a d₉₀ of from about 2 microns to about 50 microns, (ii) lactose particles having a d₉₀ of from about 50 microns to about 185 microns, and (iii) mixtures of lactose particles having a d₉₀ of from about 2 microns to about 50 microns and lactose particles having a d₉₀ of from about 50 microns to about 185 microns thereof.

In a specific embodiment, the inhalable grades of lactose is commercially available as dry powder compositions such as Respitose® SV010, Respitose® SV003, Respitose® ML006, InhaLac 70, InhaLac 230, Lactohale® 300, Lactohale® 201, Lactohale® 200, Lactose Monohydrate Inhalation 40M, and Lactose Anhydrous 120M. Respitose and Lactohale products are available from DFE Pharma of Goch, Germany. Inhalac products are available from Meggle Group Wasserburg of Wasserburg, Germany. Lactose 40M and 120M are available from Kerry Group of Beloit, WI, USA.

Yet another embodiment is an inhalable dry powder composition comprising a therapeutically effective dosage amount of compound (I) or a pharmaceutically acceptable salt thereof, and a lactose component, where the lactose component comprises a mixture of (i) lactose particles having a d₉₀ of less than about 50 microns and (ii) lactose particles having a d₉₀ of less than about 200 microns.

One embodiment is an inhalable dry powder composition comprising a therapeutically effective dosage amount of compound (I) or a pharmaceutically acceptable salt thereof, and a lactose component, wherein the lactose component comprises a mixture of Respitose® ML006 (lactose monohydrate having a d₁₀ of 0.5 microns to 10 microns, d₅₀ of 10 microns to 30 microns, d₉₀ of 30 microns to 60 microns) and Respitose® SV010 (lactose having a d₁₀ of from about 35 to about 65 microns, d₅₀ of from about 95 to about 125 microns, and d₉₀ of from about 160 to about 190 microns).

Yet another embodiment is An inhalable dry powder composition comprising a therapeutically effective dosage amount of compound (I) or a pharmaceutically acceptable salt thereof, in the range of from about 0.1 mg to about 20 mg and a lactose component.

Yet another embodiment is an inhalable dry powder composition comprising a therapeutically effective dosage amount of compound (I) or a pharmaceutically acceptable salt thereof, in the range of from about 0.1 mg to about 20 mg and a lactose component having aerodynamic diameter of less than about 60 microns.

Yet another embodiment is an inhalable dry powder composition comprising a therapeutically effective dosage amount of compound (I) or a pharmaceutically acceptable salt thereof, in the range of from about 0.1 mg to about 20 mg and a lactose component having aerodynamic diameter of less than about 200 microns.

Yet another embodiment is an inhalable dry powder composition comprising a therapeutically effective dosage amount of compound (I) or a pharmaceutically acceptable salt thereof, in the range of from about 0.1 mg to about 20 mg and a lactose component, wherein the lactose component is in the amount of from 0 mg to about 19.9 mg.

Yet another embodiment is an inhalable dry powder composition comprising a therapeutically effective dosage amount of compound (I) or a pharmaceutically acceptable salt thereof, in the range of from about 0.1 mg to about 20 mg and a lactose component, wherein the lactose component comprises lactose particles having d90 particle size less than about 60 microns and in the amount of from 0 mg to about 19.9 mg.

Yet another embodiment is an inhalable dry powder composition comprising 0.5 mg of compound (I) or a pharmaceutically acceptable salt thereof, and a lactose component, wherein the lactose component comprises lactose particles having a d90 particle size less than about 60 microns and in the amount of from about 1 mg to about 19.5 mg.

Yet another embodiment is an inhalable dry powder composition comprising 1 mg of compound (I) or a pharmaceutically acceptable salt thereof, and a lactose component, wherein the lactose component comprises lactose particles having a d90 particle size less than about 60 microns and in the amount of from about 1 mg to about 19 mg.

Yet another embodiment is an inhalable dry powder composition comprising 2.5 mg of compound (I) or a pharmaceutically acceptable salt thereof, and a lactose component, wherein the lactose component comprises lactose particles having a d90 particle size less than about 60 microns and in the amount of from about 1 mg to about 17.5 mg.

Yet another embodiment is an inhalable dry powder composition comprising 5 mg of compound (I) or a pharmaceutically acceptable salt thereof, and a lactose component, wherein the lactose component comprises lactose particles having a d90 particle size less than about 60 microns and in the amount of from about 1 mg to about 15 mg.

Yet another embodiment is an inhalable dry powder composition comprising 10 mg of compound (I) or a pharmaceutically acceptable salt thereof, and a lactose component, wherein the lactose component comprises lactose particles having a d90 particle size less than about 60 microns and in the amount of from about 1 mg to about 10 mg.

Yet another embodiment is an inhalable dry powder composition comprising a therapeutically effective dosage amount of compound (I) or a pharmaceutically acceptable salt thereof, in the range of from about 0.1 mg to about 20 mg and a lactose component, wherein the lactose component comprises lactose particles having d90 particle size less than about 190 microns and in the amount of from about 1 mg to about 19.9 mg.

Yet another embodiment is an inhalable dry powder composition comprising 0.5 mg of compound (I) or a pharmaceutically acceptable salt thereof, and a lactose component, wherein the lactose component comprises lactose particles having a d90 particle size less than about 190 microns and in the amount of from about 1 mg to about 19.5 mg.

Yet another embodiment is an inhalable dry powder composition comprising 1 mg of compound (I) or a pharmaceutically acceptable salt thereof, and a lactose component, wherein the lactose component comprises lactose particles having a d90 particle size less than about 190 microns and in the amount of from about 1 mg to about 19 mg.

Yet another embodiment is an inhalable dry powder composition comprising 2.5 mg of compound (I) or a pharmaceutically acceptable salt thereof, and a lactose component, wherein the lactose component comprises lactose particles having a d90 particle size less than about 190 microns and in the amount of from about 1 mg to about 17.5 mg.

Yet another embodiment is an inhalable dry powder composition comprising 5 mg of compound (I) or a pharmaceutically acceptable salt thereof, and a lactose component, wherein the lactose component comprises lactose particles having a d90 particle size less than about 190 microns and in the amount of from about 1 mg to about 15 mg.

Yet another embodiment is an inhalable dry powder composition comprising 10 mg of compound (I) or a pharmaceutically acceptable salt thereof, and a lactose component, wherein the lactose component comprises lactose particles having a d90 particle size less than about 190 microns and in the amount of from about 1 mg to about 10 mg.

Yet another embodiment is an inhalable dry powder composition comprising a therapeutically effective dosage amount of compound (I) or a pharmaceutically acceptable salt thereof, in the range of from about 0.1 mg to about 20 mg and a lactose component, wherein the lactose component comprises i) lactose particles having aerodynamic diameter of less than about 60 and ii) lactose particles having aerodynamic diameter of less than about 200 microns.

Yet another embodiment is an inhalable dry powder composition comprising a therapeutically effective dosage amount of compound (I) or a pharmaceutically acceptable salt thereof, and a lactose component, where the lactose component comprises a mixture of (i) lactose particles having aerodynamic diameter of less than about 50 microns and (ii) lactose particles having aerodynamic diameter of less than about 200 microns.

Yet another embodiment is an inhalable dry powder composition comprising a therapeutically effective dosage amount of compound (I) or a pharmaceutically acceptable salt thereof, and a lactose component.

Yet another embodiment is an inhalable dry powder composition comprising a therapeutically effective dosage amount of compound (I) or a pharmaceutically acceptable salt thereof, and a lactose component; wherein the weight ratio between compound (I) to lactose is from about 1:1 to about 1:400.

Yet another embodiment is an inhalable dry powder composition comprising a therapeutically effective dosage amount of compound (I) or a pharmaceutically acceptable salt thereof, and a lactose component; wherein the weight ratio between compound (I) to lactose is from about 1:1 to about 1:350.

Yet another embodiment is an inhalable dry powder composition comprising a therapeutically effective dosage amount of compound (I) or a pharmaceutically acceptable salt thereof, and a lactose component; wherein the weight ratio between compound (I) to lactose is from about 1:1 to about 1:300.

Yet another embodiment is an inhalable dry powder composition comprising a therapeutically effective dosage amount of compound (I) or a pharmaceutically acceptable salt thereof, and a lactose component; wherein the weight ratio between compound (I) to lactose is from about 1:1 to about 1:250.

Yet another embodiment is an inhalable dry powder composition comprising a therapeutically effective dosage amount of compound (I) or a pharmaceutically acceptable salt thereof, and a lactose component; wherein the weight ratio between compound (I) to lactose is from about 1:1 to about 1:200.

Yet another embodiment is an inhalable dry powder composition comprising a therapeutically effective dosage amount of compound (I) or a pharmaceutically acceptable salt thereof, and a lactose component; wherein the weight ratio between compound (I) to lactose is from about 1:1 to about 1:100.

Yet another embodiment is an inhalable dry powder composition comprising a therapeutically effective dosage amount of compound (I) or a pharmaceutically acceptable salt thereof, and a lactose component; wherein the weight ratio between compound (I) to lactose is from about 1:1 to about 1:50.

Yet another embodiment is an inhalable dry powder composition comprising about 0.5 mg of compound (I) or a pharmaceutically acceptable salt thereof, and a lactose component; wherein the weight ratio between compound (I) to lactose is from about 1:10 to about 1:50.

Yet another embodiment is an inhalable dry powder composition comprising about 1 mg of compound (I) or a pharmaceutically acceptable salt thereof, and a lactose component; wherein the weight ratio between compound (I) to lactose is from about 1:1 to about 1:40.

Yet another embodiment is an inhalable dry powder composition comprising about 2.5 mg of compound (I) or a pharmaceutically acceptable salt thereof, and a lactose component; wherein the weight ratio between compound (I) to lactose is from about 1:1 to about 1:20.

Yet another embodiment is an inhalable dry powder composition comprising about 5 mg of compound (I) or a pharmaceutically acceptable salt thereof, and a lactose component; wherein the weight ratio between compound (I) to lactose is from about 1:1 to about 1:10.

Yet another embodiment is an inhalable dry powder composition comprising about 10 mg of compound (I) or a pharmaceutically acceptable salt thereof, and a lactose component; wherein the weight ratio between compound (I) to lactose is from about 1:1 to about 1:5.

Yet another embodiment, the inhalable dry powder composition having weight ratio between compound (I) to lactose is selected from 1:1, 1:2, 1:3, 1:4, 1:5. 1:6, 1:7, 1:8, 1:9, 1:10, 1:11, 1:12, 1:13, 1:14, 1:15, 1:16, 1:17, 1:18, 1:19, 1:20, 1:21, 1:22, 1:23, 1:24, 1:25, 1:26, 1:27, 1:28, 1:29, 1:30, 1:31, 1:32, 1:33, 1:34, 1:35, 1:36, 1:37, 1:38, 1:39, 1:40, 1:41, 1:42, 1:43, 1:44, 1:45, 1:46, 1:47, 1:48, 1:49, and 1:50.

Yet another embodiment, the inhalable dry powder composition having weight ratio between compound (I) to lactose is selected from 1:1, 1:1.5, 1:3, 1:7, 1:19 and 1:39.

Yet another embodiment is an inhalable dry powder composition comprising a therapeutically effective dosage amount of compound (I) or a pharmaceutically acceptable salt thereof, in the range of from about 0.1 mg to about 20 mg and one or more pharmaceutically acceptable excipient(s).

In some embodiments, the inhalable dry powder composition comprises compound (I) and Respitose ML006 in the weight ratio of 0.1:24.9 i.e., 1:250.

In some embodiments, the inhalable dry powder composition comprises compound (I) and Respitose SV010 in the weight ratio of 0.1:24.9, i.e., 1:250.

In some embodiments, the inhalable dry powder composition comprises compound (I) and Inhalac70 in the weight ratio of 0.1:24.9, i.e., 1:250.

In some embodiments, the inhalable dry powder composition comprises compound (I) and Respitose ML006 in the weight ratio of 10:15, i.e., 1:1.5.

In some embodiments, the inhalable dry powder composition comprises compound (I) and Respitose SV010 in the weight ratio of 10:15, i.e., 1:1.5.

In some embodiments, the inhalable dry powder composition comprises compound (I) and Inhalac70 in the weight ratio of 10:15, i.e., 1:1.5.

In some embodiments, the inhalable dry powder composition comprises compound (I) Respitose ML006 and Respitose SV010 in the weight ratio of 0.5:7.5:12.0 respectively, i.e., 1:39.

In some embodiments, the inhalable dry powder composition comprises compound (I) Respitose ML006 and Respitose SV010 in the weight ratio of 10.0:4.0:6.0 respectively, i.e., 1:39.

In some embodiments, the inhalable dry powder composition comprises compound (I) Respitose ML006 and Inhalac 70 in the weight ratio of 0.5:7.5:12.0 respectively, i.e., 1:39.

In some embodiments, the inhalable dry powder composition comprises compound (I) Respitose ML006, Respitose SV010 and Inhalac70 in the weight ratio of 0.5:7.5:6.0:6.0 respectively, i.e., 1:39.

In some embodiments, the inhalable dry powder composition comprises compound (I) and Inhalac230 in the weight ratio of 0.5:19.5, i.e., 1:39.

In some embodiments, the inhalable dry powder composition comprises compound (I) and Lactohale LH200 in the weight ratio of 0.5:19.5, i.e., 1:39.

In some embodiments, the inhalable dry powder composition comprises compound (I) and Lactohale LH300 in the weight ratio of 0.5:19.5, i.e., 1:39.

In some embodiments, the inhalable dry powder composition comprises compound (I) and Respitose SV003 in the weight ratio of 0.5:19.5, i.e., 1:39.

In some embodiments, the inhalable dry powder composition comprises compound (I) and Inhalac 400 in the weight ratio of 0.5:19.5, i.e., 1:39.

In some embodiments, the inhalable dry powder composition comprises compound (I) and Respitose SV014 in the weight ratio of 0.5:19.5, i.e., 1:39.

In some embodiments, the inhalable dry powder composition comprises compound (I) and Lactohale LH201 in the weight ratio of 0.5:19.5, i.e., 1:39.

In one embodiment, the inhalable dry powder composition described herein is in a capsule dosage form.

In another embodiment, the inhalable dry powder composition is in a sachet dosage form.

In one embodiment, the inhalable dry powder composition of the present invention is physically and chemically stable, and having total impurity less than about 2% w/w. The total impurity is not more than 2% or not more than 1% w/w, or not more than 0.15% w/w or not more than 0.1% w/w based on total weight of the composition.

One embodiment is an inhalable dry powder composition comprising i) a therapeutically effective dosage amount of compound (I) or a pharmaceutically acceptable salt thereof from about 0.1 mg to about 12 mg; and one or more pharmaceutically acceptable excipient(s); wherein the composition has particles with fine particle fraction in the range of from about 1% to about 99%.

The particles may have a fine particle fraction in the range of about 1% to about 99%, about 10% to about 90%, about 20% to about 80%, about 30% to about 70%, about 40% to about 60%, about 40% to about 50% or about 40% to about 55%.

In some embodiments, the particles of the composition have a fine particle fraction of from about 15% to about 60%.

In some embodiments, the particles of the composition have a fine particle fraction of at least about 40%.

In some embodiments, the particles of the composition have a fine particle fraction of at least about 50%.

In some embodiments, the particles of the composition have a fine particle fraction of at least about 60%.

In a preferred embodiment, the inhalable dry powder composition comprising i) about 0.5 mg of compound (I) or a pharmaceutically acceptable salt thereof; and one or more pharmaceutically acceptable excipient(s); wherein the composition has particles having fine particle fraction in the range of from about 25% to about 40%.

In a preferred embodiment, the inhalable dry powder composition comprising i) about 6 mg of compound (I) or a pharmaceutically acceptable salt thereof; and one or more pharmaceutically acceptable excipient(s); wherein the composition has particles having fine particle fraction in the range of from about 50% to about 70%.

In a preferred embodiment, the inhalable dry powder composition comprising i) about 10 mg of compound (I) or a pharmaceutically acceptable salt thereof; and one or more pharmaceutically acceptable excipient(s); wherein the composition has particles having fine particle fraction in the range of from about 40% to about 60%.

The fine particle fraction of the inhalable dry powder composition has an aerodynamic diameter of less than about 5 microns.

In one embodiment is an inhalable dry powder composition comprising i) a therapeutically effective dosage amount of compound (I) or a pharmaceutically acceptable salt thereof from about 0.1 mg to about 12 mg; and one or more pharmaceutically acceptable excipient(s); wherein the composition has fine particle mass in the range of from about 5 mcg to about 6000 mcg.

An another embodiment is an inhalable dry powder composition comprising i) a therapeutically effective dosage amount of compound (I) or a pharmaceutically acceptable salt thereof from about 0.5 mg to about 12 mg; and one or more pharmaceutically acceptable excipient(s); wherein the composition has fine particle mass in the range of from about 50 mcg to about 6000 mcg.

In a preferred embodiment, the inhalable dry powder composition comprising i) about 0.5 mg of compound (I) or a pharmaceutically acceptable salt thereof; and one or more pharmaceutically acceptable excipient(s); wherein the composition has fine particle mass in the range of from about 50 mcg to about 200 mcg.

In a preferred embodiment, the inhalable dry powder composition comprising i) about 1 mg of compound (I) or a pharmaceutically acceptable salt thereof; and one or more pharmaceutically acceptable excipient(s); wherein the composition has fine particle mass in the range of from about 10 mcg to about 400 mcg.

In a preferred embodiment, the inhalable dry powder composition comprising i) about 2.5 mg of compound (I) or a pharmaceutically acceptable salt thereof; and one or more pharmaceutically acceptable excipient(s); wherein the composition has fine particle mass in the range of from about 500 mcg to about 1000 mcg.

In a preferred embodiment, the inhalable dry powder composition comprising i) about 5 mg of compound (I) or a pharmaceutically acceptable salt thereof; and one or more pharmaceutically acceptable excipient(s); wherein the composition has fine particle mass in the range of from about 1000 mcg to about 3000 mcg.

In a preferred embodiment, the inhalable dry powder composition comprising i) about 10 mg of compound (I) or a pharmaceutically acceptable salt thereof; and one or more pharmaceutically acceptable excipient(s); wherein the composition has fine particle mass in the range of from about 3000 mcg to about 6000 mcg.

In another embodiment, there is provided an inhalable dry powder composition comprising i) a therapeutically effective dosage amount of compound (I) or a pharmaceutically acceptable salt thereof from about 0.5 mg to about 10 mg; and one or more pharmaceutically acceptable excipient(s); wherein the composition has Hausner ratio in the range of from about 0.1 to about 3 or preferably Hausner ratio is in the range of from about 1 to about 2.5 or more preferably Hausner ratio is in the range of from about 1.2 to 2.

In another embodiment, the inhalable dry powder composition comprising i) about 0.5 mg of compound (I) or a pharmaceutically acceptable salt thereof; and one or more pharmaceutically acceptable excipient(s); wherein the composition has Hausner ratio in the range of from about 1 to about 2.

In another embodiment, the inhalable dry powder composition comprising i) about 1 mg of compound (I) or a pharmaceutically acceptable salt thereof; and one or more pharmaceutically acceptable excipient(s); wherein the composition has Hausner ratio in the range of from about 1 to about 2.

In another embodiment, the inhalable dry powder composition comprising i) about 2.5 mg of compound (I) or a pharmaceutically acceptable salt thereof; and one or more pharmaceutically acceptable excipient(s); wherein the composition has Hausner ratio in the range of from about 1 to about 2.

In another embodiment, the inhalable dry powder composition comprising i) about 5 mg of compound (I) or a pharmaceutically acceptable salt thereof; and one or more pharmaceutically acceptable excipient(s); wherein the composition has Hausner ratio in the range of from about 1.5 to about 2.5.

In another embodiment, the inhalable dry powder composition comprising i) about 10 mg of compound (I) or a pharmaceutically acceptable salt thereof; and one or more pharmaceutically acceptable excipient(s); wherein the composition has Hausner ratio in the range of from about 1.5 to about 2.5.

In another embodiment, there is provided an inhalable dry powder composition comprising i) a therapeutically effective dosage amount of compound (I) or a pharmaceutically acceptable salt thereof from about 0.5 mg to about 10 mg; and one or more pharmaceutically acceptable excipient(s); wherein the composition has Carr index in the range of from about 30% to about 65% or more preferably the Carr index is in the range of from about 35% to about 50%.

In another embodiment, the inhalable dry powder composition comprising i) 0.5 mg of compound (I) or a pharmaceutically acceptable salt thereof from; and one or more pharmaceutically acceptable excipient(s); wherein the composition has Carr index in the range of from about 35% to about 45%.

In another embodiment, the inhalable dry powder composition comprising i) 1.0 mg of compound (I) or a pharmaceutically acceptable salt thereof from; and one or more pharmaceutically acceptable excipient(s); wherein the composition has Carr index in the range of from about 35% to about 45%.

In another embodiment, the inhalable dry powder composition comprising i) 2.5 mg of compound (I) or a pharmaceutically acceptable salt thereof from; and one or more pharmaceutically acceptable excipient(s); wherein the composition has Carr index in the range of from about 35% to about 50%.

In another embodiment, the inhalable dry powder composition comprising i) 5 mg of compound (I) or a pharmaceutically acceptable salt thereof from; and one or more pharmaceutically acceptable excipient(s); wherein the composition has Carr index in the range of from about 40% to about 50%.

In another embodiment, the inhalable dry powder composition comprising i) 10 mg of compound (I) or a pharmaceutically acceptable salt thereof from; and one or more pharmaceutically acceptable excipient(s); wherein the composition has Carr index in the range of from about 40% to about 50%.

In another embodiment, there is provided an inhalable dry powder composition comprising i) a therapeutically effective dosage amount of compound (I) or a pharmaceutically acceptable salt thereof from about 0.5 mg to about 10 mg; and one or more pharmaceutically acceptable excipient(s); wherein the composition has bulk density in the range of from about 0.08 g/mL to about 1 g/mL or more preferably bulk density in the range of from about 0.2 g/mL to about 0.7 g/mL.

In another embodiment, the inhalable dry powder composition comprising i) 0.5 mg of compound (I) or a pharmaceutically acceptable salt thereof; and one or more pharmaceutically acceptable excipient(s); wherein the composition has bulk density in the range of from about 0.1 g/mL to about 1 g/mL.

In another embodiment, the inhalable dry powder composition comprising i) 1 mg of compound (I) or a pharmaceutically acceptable salt thereof; and one or more pharmaceutically acceptable excipient(s); wherein the composition has bulk density in the range of from about 0.1 g/mL to about 1 g/mL.

In another embodiment, the inhalable dry powder composition comprising i) 2.5 mg of compound (I) or a pharmaceutically acceptable salt thereof; and one or more pharmaceutically acceptable excipient(s); wherein the composition has bulk density in the range of from about 0.1 g/mL to about 0.8 g/mL.

In another embodiment, the inhalable dry powder composition comprising i) 5 mg of compound (I) or a pharmaceutically acceptable salt thereof; and one or more pharmaceutically acceptable excipient(s); wherein the composition has bulk density in the range of from about 0.1 g/mL to about 0.8 g/mL.

In another embodiment, the inhalable dry powder composition comprising i) 10 mg of compound (I) or a pharmaceutically acceptable salt thereof; and one or more pharmaceutically acceptable excipient(s); wherein the composition has bulk density in the range of from about 0.08 g/mL to about 0.8 g/mL.

In another embodiment, there is provided an inhalable dry powder composition comprising i) a therapeutically effective dosage amount of compound (I) or a pharmaceutically acceptable salt thereof from about 0.1 mg to about 12 mg; and one or more pharmaceutically acceptable excipient(s); wherein the composition has tap density in the range of from about 0.1 g/mL to about 1.5 g/mL or more preferably tap density in the range of from 0.2 g/mL to about 1.25 g/mL.

In another embodiment, the inhalable dry powder composition comprising i) 0.5 mg of compound (I) or a pharmaceutically acceptable salt thereof; and one or more pharmaceutically acceptable excipient(s); wherein the composition has tap density in the range of from about 0.5 g/mL to about 1.5 g/mL.

In another embodiment, the inhalable dry powder composition comprising i) 1 mg of compound (I) or a pharmaceutically acceptable salt thereof; and one or more pharmaceutically acceptable excipient(s); wherein the composition has tap density in the range of from about 0.5 g/mL to about 1.5 g/mL.

In another embodiment, the inhalable dry powder composition comprising i) 2.5 mg of compound (I) or a pharmaceutically acceptable salt thereof; and one or more pharmaceutically acceptable excipient(s); wherein the composition has tap density in the range of from about 0.5 g/mL to about 1.5 g/mL.

In another embodiment, the inhalable dry powder composition comprising i) 5 mg of compound (I) or a pharmaceutically acceptable salt thereof; and one or more pharmaceutically acceptable excipient(s); wherein the composition has tap density in the range of from about 0.4 g/mL to about 1.2 g/mL.

In another embodiment, the inhalable dry powder composition comprising i) 10 mg of compound (I) or a pharmaceutically acceptable salt thereof; and one or more pharmaceutically acceptable excipient(s); wherein the composition has tap density in the range of from about 0.2 g/mL to about 1 g/mL.

In another embodiment, there is provided an inhalable dry powder composition comprising i) a therapeutically effective dosage amount of compound (I) or a pharmaceutically acceptable salt thereof from about 0.1 mg to about 12 mg; and one or more pharmaceutically acceptable excipient(s); wherein MMAD of compound (I) in said composition is in the range of from about 1 micron to about 6 microns or more preferably MMAD of compound (I) in said composition is in the range of from about 1 micron to about 6 microns.

In another embodiment, there is provided an inhalable dry powder composition comprising i) a therapeutically effective dosage amount of compound (I) or a pharmaceutically acceptable salt thereof from about 0.1 mg to about 12 mg; and one or more pharmaceutically acceptable excipient(s); wherein MMAD of compound (I) in said composition is in the range of from about 1 micron to about 6 microns or more preferably MMAD of compound (I) in said composition is in the range of from about 2 micron to about 6 microns.

In an embodiment, the inhalable dry powder composition comprising a therapeutically effective dose of compound (I) or a pharmaceutically acceptable salt thereof; and one or more pharmaceutically acceptable excipient(s); wherein the composition is characterized by fine particle fraction of the composition having aerodynamic diameter of less than about 5 microns.

In another embodiment, the inhalable dry powder composition comprising i) 0.5 mg of compound (I) or a pharmaceutically acceptable salt thereof; and one or more pharmaceutically acceptable excipient(s); wherein MMAD of compound (I) in said composition is in the range of from about 1 micron to about 6 microns.

In another embodiment, the inhalable dry powder composition comprising i) 1 mg of compound (I) or a pharmaceutically acceptable salt thereof; and one or more pharmaceutically acceptable excipient(s); wherein MMAD of compound (I) in said composition is in the range of from about 1 micron to about 6 microns.

In another embodiment, the inhalable dry powder composition comprising i) 2.5 mg of compound (I) or a pharmaceutically acceptable salt thereof; and one or more pharmaceutically acceptable excipient(s); wherein MMAD of compound (I) in said composition is in the range of from about 1 micron to about 6 microns.

In another embodiment, the inhalable dry powder composition comprising i) 5 mg of compound (I) or a pharmaceutically acceptable salt thereof; and one or more pharmaceutically acceptable excipient(s); wherein MMAD of compound (I) in said composition is in the range of from about 1 micron to about 6 microns.

In another embodiment, the inhalable dry powder composition comprising i) 10 mg of compound (I) or a pharmaceutically acceptable salt thereof; and one or more pharmaceutically acceptable excipient(s); wherein MMAD of compound (I) in said composition is in the range of from about 1 micron to about 6 microns.

In an embodiment, an inhalable dry powder composition comprising i) a therapeutically effective dosage amount of compound (I) or a pharmaceutically acceptable salt thereof; ii) lactose particles having a d₉₀ particle size less than about 50 microns; iii) lactose particles having a d₉₀ particle size less than about 200 microns.

Another embodiment is an inhalable dry powder composition comprising i) a therapeutically effective dosage amount of compound (I) or a pharmaceutically acceptable salt thereof; ii) lactose particles having a d₉₀ particle size less than about 50 microns; iii) lactose particles having a d₉₀ particle size less than about 200 microns, wherein the average particle size of compound (I) or a pharmaceutically acceptable salt thereof is from about 0.1 microns to about 10 microns.

Yet another embodiment is an inhalable dry powder composition comprising i) a therapeutically effective dosage amount of compound (I) or a pharmaceutically acceptable salt thereof; ii) lactose particles having a d₉₀ particle size less than about 50 microns; iii) lactose particles having a d₉₀ particle size less than about 200 microns, wherein the average particle size of compound (I) or a pharmaceutically acceptable salt thereof is from about 0.1 micron to about 10 microns.

In a specific embodiment is an inhalable dry powder composition comprising i) from about 0.5 mg to about 10 mg of compound (I) or a pharmaceutically acceptable salt thereof; ii) from about 3 mg to about 8 mg of lactose particles having a d₉₀ particle size less than about 50 microns; iii) from about 5 to about 13 mg of lactose particles having a d₉₀ particle size less than about 200 microns.

In a specific embodiment is an inhalable dry powder composition comprising i) about 0.5 mg of compound (I) or a pharmaceutically acceptable salt thereof; ii) about 7.5 mg of lactose particles having a d₉₀ particle size less than about 50 microns; iii) about 12 mg of lactose particles having a d₉₀ particle size less than about 200 microns, wherein the weight ratio between compound (I) to lactose is from about 1:1 to about 1:50.

In a specific embodiment is an inhalable dry powder composition comprising i) about 1 mg of compound (I) or a pharmaceutically acceptable salt thereof; ii) about 7 mg of lactose particles having a d₉₀ particle size less than about 50 microns; iii) about 12 mg of lactose particles having a d₉₀ particle size less than about 200 microns, wherein the weight ratio between compound (I) to lactose is from about 1:1 to about 1:50.

In a specific embodiment is an inhalable dry powder composition comprising i) about 2.5 mg of compound (I) or a pharmaceutically acceptable salt thereof; ii) about 5.5 mg of lactose particles having a d₉₀ particle size less than about 50 microns; iii) about 12 mg of lactose particles having a d₉₀ particle size less than about 200 microns, wherein the weight ratio between compound (I) to lactose is from about 1:1 to about 1:50.

In a specific embodiment is an inhalable dry powder composition comprising i) about 5 mg of compound (I) or a pharmaceutically acceptable salt thereof; ii) about 3 mg of lactose particles having a d₉₀ particle size less than about 50 microns; iii) about 12 mg of lactose particles having a d₉₀ particle size less than about 200 microns, wherein the weight ratio between compound (I) to lactose is from about 1:1 to about 1:50.

In a specific embodiment is an inhalable dry powder composition comprising i) about 10 mg of compound (I) or a pharmaceutically acceptable salt thereof; ii) about 3.4 mg of lactose particles having a d₉₀ particle size less than about 50 microns; iii) about 6.6 mg of lactose particles having a d₉₀ particle size less than about 200 microns, wherein the weight ratio between compound (I) to lactose is from about 1:1 to about 1:50.

In a specific embodiment, the present invention relates to an inhalable dry powder composition comprising a therapeutically effective dosage amount of compound (I) or a pharmaceutically acceptable salt thereof, in the range of from about 0.1 mg to about 20 mg and a lactose component, wherein the lactose component comprises lactose particles having a d90 particle size less than about 60 microns and in the amount of from about 1 mg to about 19.9 mg.

In one embodiment, the present invention relates to a method of preventing, inhibiting, suppressing, and/or treating at least one pulmonary disorder or a symptom of a pulmonary infection in a subject in need thereof.

The pulmonary disease can be any disorder, disease of respiratory system characterized by airflow obstruction or limitation, or a symptom occurring in lung or respiratory system due to pulmonary infection.

In an embodiment, the present invention relates to a method of preventing, inhibiting, suppressing, and/or treating at least one pulmonary disease(s) characterized by airflow obstruction or limitation, or a symptom occurring in pulmonary system, including but not limited to asthma, emphysema, bronchitis, COPD, sinusitis, respiratory depression, reactive airways dysfunction syndrome (RADS), acute respiratory distress syndrome (ARDS), irritant induced asthma, occupational asthma, symptoms of pulmonary infection(s), sensory hyper-reactivity, airway (or pulmonary) inflammation, multiple chemical sensitivity, aid in smoking cessation therapy, pneumonia, bronchitis, influenza, SARS, SARS-CoV-2 (COVID-19), tuberculosis, and whooping cough (pertussis), and the like in a subject in need thereof.

Yet another embodiment is a method of preventing, inhibiting, suppressing, and/or treating at least one pulmonary disease in a subject in need thereof comprising administering compound (I) or a pharmaceutically acceptable salt thereof to the subject via inhalation.

Yet another embodiment is a method of preventing, inhibiting, suppressing, and/or treating at least one pulmonary disease in a subject in need thereof comprising administering a therapeutically effective dosage amount of compound (I) or a pharmaceutically acceptable salt thereof to the subject.

Yet another embodiment is a method of treating a symptom of a pulmonary infection.

Pulmonary infection, including viral, bacterial, or fungal infection, can also lead to inflammation and/or scarring of the lungs, the pleural cavity that surrounds the lungs, the alveoli, and/or the bronchial passages. In particular, pneumonia, influenza, SARS, SARS-CoV-2 (COVID-19), tuberculosis, and whooping cough (pertussis) can all lead to chronic lung problems due to inflammation and scarring in the lungs.

In some embodiments, the pulmonary infection(s) includes viral, bacterial, or fungal infection. The symptoms of pulmonary infection(s) is mild to severe breathing problem, dry or productive cough, chest pain, whooping cough and/or sore throat.

Yet another embodiment is a method of treating symptom(s) of pulmonary infection(s) in a subject in the need thereof, comprising administering a therapeutically effective dosage amount of compound (I) or a pharmaceutically acceptable salt thereof to the subject via inhalation.

Yet another embodiment, the inhalable dry powder composition comprising a therapeutically effective dosage amount of compound (I) or a pharmaceutically acceptable salt thereof for use in preventing, inhibiting, suppressing, and/or treating at least one pulmonary disease.

The administration of the inhalable dry powder composition via inhalation may be performed with any suitable device, such as a dry powder inhaler. It may also be possible that dry powder composition of the present invention may include meter dose inhaler. The dry powder inhaler may be portable or non-portable, and the inhaler may be designed for single use or multiple use, in other words the dry powder inhaler can be re-usable or a disposable inhaler, specifically a dry powder inhaler of the present invention comprising unit dose container, i.e., single use and single dose inhaler.

The dry powder inhaler to be used for dry powder composition of the present invention may disperse the composition at a rate between about 30 L/minute and about 100 L/minute. In a specific embodiment, the flow rate from dry powder inhaler administers the composition between about 30 L/minute to about 80 L/minute, 30 L/minute to about 70 L/minute, 40 L/minute to about 90 L/minute, 30 L/minute to about 60 L/minute, 40 L/minute to about 70 L/minute, 50 L/minute to about 70 L/minute, 30 L/minute, 40 L/minute, 50 L/minute, 60 L/minute or 70 L/minute. Administration at these rates can achieve a substantially uniform deposition profile across all impaction stages.

Yet another embodiment is a method of preventing, inhibiting, suppressing, and/or treating COPD in a subject in need thereof comprising administering a therapeutically effective amount of compound (I) or a pharmaceutically acceptable salt thereof to the subject via inhalation.

Yet another embodiment is a method of preventing, inhibiting, suppressing, and/or treating COPD in a subject in need thereof comprising administering a therapeutically effective dosage amount of compound (I) or a pharmaceutically acceptable salt thereof to the subject via inhalation, wherein the method provides a therapeutically effective amount of compound (I) to the subject.

Yet another embodiment is a method of preventing, inhibiting, suppressing, and/or treating COPD in a subject in need thereof comprising administering a therapeutically effective dosage amount of compound (I) or a pharmaceutically acceptable salt thereof selected from about 0.1 mg to about 12 mg to the subject via inhalation, wherein the method provides a therapeutically effective amount of compound (I) to the subject.

The IC₅₀ value is believed to be a measure of the effectiveness of a compound in inhibiting biological or biochemical function. This quantitative measure generally indicates molar concentration of a particular compound (or substance) that is needed to inhibit a given biological process by half. In other words, it is the half maximal (50%) inhibitory concentration (IC) of the compound. The IC₅₀ of a drug compound can be determined by constructing a concentration-response curve so as to examine the effect of different concentrations of inhibitor on reversing agonist activity. The IC₅₀ values can be calculated for a given inhibitor by determining the concentration needed to inhibit half of the maximum biological response of the agonist. These values can be used to compare the potency of two inhibitors.

The therapeutically effective amount of compound (I) or a pharmaceutically acceptable substance is less than about 20 μM or less than about 10 μM, or about 6 μM or about 3 μM or about 1 μM or less than 1000 nM or less than about 900 nM, or less than about 800 nM, or less than about 700 nM, or less than about 600 nM, or less than about 500 nM.

The therapeutically effective amount of compound (I) of a pharmaceutically acceptable substance is from about 20 μM to about 0.1 nM.

Specifically, in an embodiment, the present invention relates to a method of preventing, inhibiting, suppressing, and/or treating at least one pulmonary disease in a subject by inhibiting IL-17 in a subject, said method comprising administering to the subject a therapeutically effective amount of Compound I or a pharmaceutically acceptable salt. The therapeutically effective amount of Compound (I) or a pharmaceutically acceptable salt may range from about 20 μM to about 0.1 nM.

In a specific embodiment, the therapeutically effective amount of compound (I) of a pharmaceutically acceptable substance is from about 1 μM to about 0.1 nM, when ex-vivo evaluation of Compound (I) for IL-17 inhibition is conducted from Bronchoalveolar lavage (BAL) cells from COPD patients.

In a specific embodiment, the therapeutically effective amount of compound (I) of a pharmaceutically acceptable substance is from about 700 nM to about 10 nM, when ex-vivo evaluation of Compound (I) for IL-17 inhibition is conducted from Bronchoalveolar lavage (BAL) cells from COPD patients.

In a specific embodiment, the therapeutically effective amount of compound (I) of a pharmaceutically acceptable substance is from about 500 nM to about 50 nM, when ex-vivo evaluation of Compound (I) for IL-17 inhibition is conducted from Bronchoalveolar lavage (BAL) cells from COPD patients.

In a specific embodiment, the therapeutically effective amount of compound (I) of a pharmaceutically acceptable substance is from about 20 μM to about 0.1 nM, when ex-vivo evaluation of Compound (I) for IL-17 inhibition is conducted from peripheral blood from COPD patients.

In a specific embodiment, the therapeutically effective amount of compound (I) of a pharmaceutically acceptable substance is from about 10 μM to about 0.1 nM, when ex-vivo evaluation of Compound (I) for IL-17 inhibition is conducted from peripheral blood from COPD patients.

In a specific embodiment, the therapeutically effective amount of compound (I) of a pharmaceutically acceptable substance is from about 1 μM to about 10 nM, when ex-vivo evaluation of Compound (I) for IL-17 inhibition is conducted from peripheral blood from COPD patients.

In a specific embodiment, the therapeutically effective amount of compound (I) of a pharmaceutically acceptable substance is from about 500 nM to about 50 nM, when ex-vivo evaluation of Compound (I) for IL-17 inhibition is conducted from peripheral blood from COPD patients.

In an embodiment, the present invention relates to a method of preventing, inhibiting, suppressing, and/or treating at least one pulmonary disease mediated by IL-17 blockade is subject in need thereof, wherein the method comprising administering to the subject an therapeutically effective amount of compound (I) or a pharmaceutically acceptable salt thereof.

In a specific embodiment, the present invention relates to a method of preventing, inhibiting, suppressing, and/or treating at least one pulmonary disease mediated by IL-17 blockade is subject in need thereof, wherein the method comprising administering to the subject an therapeutically effective amount of compound (I) or a pharmaceutically acceptable salt thereof; wherein the therapeutically effective amount is from about 20 μM to about 0.1 nM.

In a specific embodiment, the present invention relates to a method of preventing, inhibiting, suppressing, and/or treating at least one pulmonary disease mediated by IL-17 blockade is subject in need thereof, wherein the method comprising administering to the subject an therapeutically effective amount of compound (I) or a pharmaceutically acceptable salt thereof; wherein the therapeutically effective amount is from about 10 μM to about 1 nM.

In a specific embodiment, the present invention relates to a method of preventing, inhibiting, suppressing, and/or treating at least one pulmonary disease mediated by IL-17 blockade is subject in need thereof, wherein the method comprising administering to the subject an therapeutically effective amount of compound (I) or a pharmaceutically acceptable salt thereof; wherein the therapeutically effective amount is from about 1 μM to about 10 nM.

In a specific embodiment, the present invention relates to a method of preventing, inhibiting, suppressing, and/or treating at least one pulmonary disease mediated by IL-17 blockade is subject in need thereof, wherein the method comprising administering to the subject an therapeutically effective amount of compound (I) or a pharmaceutically acceptable salt thereof; wherein the therapeutically effective amount is from about 500 μM to about 50 nM, when ex-vivo evaluation of Compound (I) for IL-17 inhibition is conducted from Bronchoalveolar lavage (BAL) cells from COPD patients.

In a specific embodiment, the present invention relates to a method of preventing, inhibiting, suppressing, and/or treating at least one pulmonary disease mediated by IL-17 blockade is subject in need thereof, wherein the method comprising administering to the subject an therapeutically effective amount of compound (I) or a pharmaceutically acceptable salt thereof; wherein the therapeutically effective amount is from about 500 μM to about 50 nM, when ex-vivo evaluation of Compound (I) for IL-17 inhibition is conducted from peripheral blood from COPD patients.

In some embodiments, use of compound (I) or a pharmaceutically acceptable salt thereof in inhibition of IL-17 that mediates a pulmonary disease condition in the manufacture of a medicament for preventing, inhibiting, suppressing, and/or treating of the pulmonary disease in a subject in need thereof.

In some embodiments, use of compound (I) or a pharmaceutically acceptable salt thereof in inhibition of IL-17 that mediates a pulmonary disease condition in the manufacture of a medicament for preventing, inhibiting, suppressing, and/or treating of the pulmonary disease in a subject in need thereof; wherein the subject is administered a therapeutically effective amount of compound (I) or a pharmaceutically acceptable salt thereof.

In some embodiments, use of compound (I) or a pharmaceutically acceptable salt thereof in inhibition of IL-17 that mediates a pulmonary disease condition in the manufacture of a medicament for preventing, inhibiting, suppressing, and/or treating of the pulmonary disease in a subject in need thereof; wherein the subject is administered a therapeutically effective amount of compound (I) or a pharmaceutically acceptable salt thereof from about 500 nM to about 50 nM, when ex-vivo evaluation of Compound (I) for IL-17 inhibition is conducted from Bronchoalveolar lavage (BAL) cells from COPD patients.

In some embodiments, use of compound (I) in inhibition of IL-17 that mediates a pulmonary disease condition in the manufacture of a medicament for preventing, inhibiting, suppressing, and/or treating of the pulmonary disease in a subject in need thereof; wherein the subject is administered a therapeutically effective amount of compound (I) or a pharmaceutically acceptable salt thereof from about 500 nM to about 50 nM, when ex-vivo evaluation of Compound (I) for IL-17 inhibition is conducted from peripheral blood from COPD patients.

Yet another embodiment is a method of administering inhalable dry powder composition comprising a therapeutically effective dosage amount of compound (I) or a pharmaceutically acceptable salt thereof to the subject; wherein the method comprises administration of a unit dosage rapidly with one or two, small volume breaths as needed.

Yet another embodiment is a method of administering inhalable dry powder composition comprising from about 0.1 mg to about 12 mg of compound (I) or a pharmaceutically acceptable salt thereof to the subject; wherein the method comprises administration of a unit dosage rapidly with one or two, small volume breaths as needed.

Yet another embodiment, use of an inhalable dry powder composition comprising a therapeutically effective dosage amount of compound (I) or a pharmaceutically acceptable salt thereof in the manufacture of a medicament for preventing, inhibiting, suppressing, and/or treating at least one pulmonary disease.

In an embodiment, the present invention relates to a process for preparing an inhalable composition. The process for preparing inhalable dry powder composition involves any suitable processes known to a person skilled in the art for preparing inhalable dry powder composition such as
i) simple blending of compound (I) and diluent;
ii) co-micronization of compound (I) with diluent using suitable milling process such as jet milling/ball milling of compound (I) and lactose together with or without lubricant like magnesium stearate;
iii) conditioning of compound (I) with lactose, such as exposure to moisture to encourage re-crystallisation of the material without the formation of hard agglomerates;
iv) blending compound (I) and two diluents that are coarser and finer respectively, the diluent mixture is prepared by still two diluents are added, For example, two components are preferably sieved in alternately in 15 to 45, most preferably 20 to 40 layers each, and the mixing of the two diluent may take place while the two components are still being added;
v) Process of blending may preferably be carried out by first metering the excipient into the appropriate mixing container. Then, shortly after the metering of the excipient has started, the continuous feeding (metering) of the active substance may be started

Yet another embodiment is a process for preparing an inhalable composition comprising compound (I) or a pharmaceutically acceptable salt thereof comprising: i) mixing a therapeutically effective dosage amount of compound (I) or a pharmaceutically acceptable salt thereof with one or more pharmaceutically acceptable excipient(s), and ii) filling the blend in a capsule/blister/sachet dosage form meant for a unit dose dry powder inhaler.

Yet another embodiment, the process for preparing an inhalable dry powder composition comprising compound (I) or a pharmaceutically acceptable salt thereof, comprises steps of: i) blending compound (I) or a pharmaceutically acceptable salt thereof with a diluent selected from one or more lactose component; and ii) optionally preparing final blend comprising compound (I) or a pharmaceutically acceptable salt thereof and the lactose component; wherein the blending step is selected from i) simple blending, ii) co-milling of compound (I) with lactose component, iii) conditioning of compound (I) with or without lactose component; and v) metering of lactose component in blending process.

In some embodiments, the blending process of the present invention is simple blending of compound (I) with one or more lactose component at least for 10 minutes to 1 hour with the speed of 20 to 40±10 RPM.

In some embodiments, the blending process of the present invention is simple blending of compound (I) with one or more lactose component at least for 10 minutes to 1 hour with the speed of 30 to 35±10 RPM.

In some embodiments, the blending process of the present invention is simple blending of compound (I) with finer lactose component having d₉₀ particle size less than about 100 microns, and the final blending is prepared by blending mixture of step i) with a lactose component with d₉₀ particle size more than about 100 microns.

In some embodiments, the blending process of the present invention involves addition of compound (I) to the lactose component in staged or metering method.

In some embodiments, the process of the present invention may comprise conditioning of compound (I) or a pharmaceutically acceptable salts thereof for a suitable period of time such as 10 minutes to 1, 2, 3 years to avoid hard agglomerates. For example, the conditioning method, according to the present invention, involves exposing compound (I) to the controlled humid conditions 30-95 RH, preferably 40-95 RH, 45-95 RH or 50-90 RH, preferably micronized powder form of compound (I) is placed in a tray and preferably agitated or turned to ensure that all of the particles are equally exposed to the humid atmosphere. The turning or agitating also helps to avoid or reduce agglomeration of the particles during the conditioning process. The conditioning preferably takes place over a period of at least about 10 minutes, at least about 20 minutes, at least about 30 minutes, at least about 40 minutes, at least about 50 minutes, at least about 1 hour, at least about 2, 3, 4, 5, 6, 8, 10, 12, 14, 18, 24, 36 or 48 hours.

In some embodiments, the blending process involves co-milling of compound (I) or a pharmaceutically acceptable salt thereof with one or more lactose component(s).

Yet another embodiment is a process for preparing an inhalable dry powder composition comprising compound (I) or a pharmaceutically acceptable salt thereof comprising: i) mixing a therapeutically effective dosage amount of compound (I) or a pharmaceutically acceptable salt thereof with lactose particles having a d₉₀ particle size less than about 60 microns to prepare a blend; and ii) mixing the blend of step i) with lactose particles having a d₉₀ particle size more than about 100 microns to prepare a final inhalation powder comprising compound (I) or a pharmaceutically acceptable salt thereof.

Yet another embodiment is a process for preparing an inhalable dry powder composition comprising compound (I) or a pharmaceutically acceptable salt thereof, comprises: i) mixing a therapeutically effective dosage amount of compound (I) or a pharmaceutically acceptable salt thereof with lactose particles having a d₉₀ particle size less than about 60 microns, preferably less than about 30 microns, more preferably less than about 20 microns; and ii) blending the mixture of step (i) with lactose particles having a d₉₀ particle size in the range of about less than about 200 microns, preferably more than about 160 microns to prepare a final inhalable composition comprising compound (I) in a capsule/blister/sachet dosage form meant for unit dose dry powder inhaler.

Yet another embodiment is a process for preparing an inhalable dry powder composition comprising compound (I) or a pharmaceutically acceptable salt thereof, comprising mixing a therapeutically effective dosage amount of compound (I) or a pharmaceutically acceptable salt thereof with lactose particles having a d₉₀ particle size less than about 60 microns, preferably less than about 30 microns, more preferably less than about 20 microns; and ii) blending the mixture of step (i) with lactose particles having a d₉₀ particle size in the range of about 150 microns to about 200 microns to prepare a final inhalable composition comprising compound (I).

In another embodiment, the process for preparing an inhalable dry powder composition comprising compound (I) or a pharmaceutically acceptable salt thereof, comprises:
i) Sifting compound (I) with a part quantity of lactose monohydrate (Respitose ML006) using mechanical sifter through #40 American Society for Testing and Materials (ASTM);
ii) blending the mixture of step i) using a powder blender for 15 minutes at 20 to 40±10 rpm;
iii) remaining quantity of lactose monohydrate (Respitose ML006) is sifted through #40 ASTM and mix with blend of step ii);
iv) Mixing pre-sifted lactose monohydrate (Respitose SV010) to blender and mix the powder from step iii) in a powder blender blend for 15 minutes at 30±2 rpm; and
v) capsule filing of the blend from step iv).

In some embodiments, the final blend is filled into a hard gelatin capsule (for example e.g. size 3 with volume of 270 μl).

The process for preparing a dry powder composition of the present invention comprises process controls such as blend uniformity analysis.

In another embodiment, there is provided an inhalable dry powder composition comprising from about 1% to about 70% by weight of compound (I) or from about 1% to about 60% by weight of compound (I) or a pharmaceutically acceptable salt thereof; wherein the composition has fine particle mass in the range of from about 5 mcg to about 6000 mcg; wherein said composition is prepared by a method comprising: i) mixing a therapeutically effective dosage amount of compound (I) or a pharmaceutically acceptable salt thereof with one or more pharmaceutically acceptable excipient(s).

In another embodiment, there is provided an inhalable dry powder composition comprising from about 1% to about 70% by weight of compound (I) or from about 30% to about 60% by weight of compound (I) or a pharmaceutically acceptable salt thereof; wherein the composition has fine particle mass in the range of from about 3000 mcg to about 5000 mcg; wherein said composition is prepared by a method comprising: i) mixing a therapeutically effective dosage amount of compound (I) or a pharmaceutically acceptable salt thereof with one or more pharmaceutically acceptable excipient(s).

The following examples are provided to enable one skilled in the art to practice the invention and are merely illustrative of the invention. The examples should not be read as limiting the scope of the invention.

### EXAMPLES

### Example 1: Inhalable Dry Powder Composition

TABLE 1

| S. | | mg/unit | | |
|---|---|---|---|---|
| No | Ingredients | 1A | 1B | 1C |
| 1 | Compound (I) | 6 | 0.5 | 10 |
| 2 | Lactose monohydrate | 0 | 6 | 4 |
| | (Respitose ML006) | | | |
| 3 | Lactose monohydrate | 0 | 13.5 | 6 |
| | (Respitose SV010) | | | |
| 4 | Total | 6 | 20 | 20 |
| | | In | In | In |
| | | capsule | capsule | capsule |

Process of preparation: i) A blend of compound (I) and lactose monohydrate (Respitose ML006) are prepared by mixing; ii) the blend from step i) is mixed with lactose monohydrate (Respitose SV010); and iii) filling the blend from step ii) in a capsule/blister/sachet to prepare the final dosage form in unit dose dry powder inhaler.

### Example 2: In Vitro Drug Deposition Study

It was observed that compound (I) is compatible with different commercially available inhalation grades of lactose monohydrate including Respitose ML006, Respitose SV010, Lactohale 201, Lactohale 200, Inhalac 230, Inhalac 70. Lactose Monohydrate is selected as a diluent for compound (I) (micronized) with the particle size d₉₀ of ≤5 microns.

Formulation development studies were conducted to demonstrate that the drug compound (I) is reproducibly delivered using Instahaler P monodose inhaler device and is able to be deposited in the lungs. The delivered dose was determined by the delivered dose uniformity (DDU) method. An Andersen cascade impactor (ACI) was used to evaluate the deposition of the emitted dose.

TABLE 2

| | | | Fine | | | | |
|---|---|---|---|---|---|---|---|
| | | | particle | | Fine | | |
| | | | mass/ | Delivered | particle | | |
| | | | dose | Dose | fraction | MMAD | GSD |
| Compositions | Strength | | (mcg) | (mcg) | (%) | (microns) | (%) |
| Example 1A | 6.0 | mg | 1650.60 | 2759.20 | 59.82 | 3.0164 | 1.8997 |
| Example 1C | 10.0 | mg | 3552.11 | 8263.76 | 42.98 | 3.0039 | 2.0814 |
| Example 1B | 0.5 | mg | 152.23 | 404.99 | 37.59 | 3.2803 | 2.3189 |

Conclusion: Compositions of example 1A, 1B and 1C had shown the result shown in the TABLE 2.

### Example 3: Inhalable Dry Powder Compositions Filled-In Capsule

TABLE 3

| | mg/capsule | | | | |
|---|---|---|---|---|---|
| Ingredients | 3A | 3B | 3C | 3D | 3E |
| Compound (I) | 0.5 | 1.0 | 2.5 | 5.0 | 10.0 |
| Lactose Monohydrate | 7.5 | 7.0 | 5.5 | 3.0 | 3.4 |
| (Respitose ML006) | | | | | |
| Lactose Monohydrate | 12.0 | 12.0 | 12.0 | 12.0 | 6.6 |
| (Respitose SV010) | | | | | |
| EHG Cap Size ‘3’ | 1 | 1 | 1 | 1 | 1 |
| White/White (Opaque) | capsule | capsule | capsule | capsule | capsule |

The manufacturing process for dry powder compositions were similar to process described in example 1. The compositions of example 3 were evaluated for parameters of dry powder inhalation compositions.

TABLE 4

| | | | Water | Car | | Fine | | | |
|---|---|---|---|---|---|---|---|---|---|
| | BD | TD | Content | Index | Housner | particle | | | |
| Compositions | (g/ml) | (g/ml) | (%) | (%) | Ratio | mass/dose | | MMAD | GSD |
| 3A | 0.615 | 1.07 | 5.45 | 42.52 | 1.73 | 123 | mcg | 4.508 | 2.114 |
| 3B | 0.546 | 0.964 | 5.33 | 43.36 | 1.77 | 283 | mcg | 4.271 | 2.054 |
| 3C | 0.506 | 0.906 | 4.94 | 44.15 | 1.79 | 744 | mcg | 3.613 | 2.116 |
| 3D | 0.428 | 0.824 | 4.69 | 48.06 | 1.93 | 2048 | mcg | 3.011 | 2.154 |
| 3E | 0.312 | 0.594 | 3.18 | 47.47 | 1.9 | 4217 | mcg | 3.103 | 2.122 |

### Example 4: Inhalable Dry Powder Compositions

TABLE 5

| | mg/ capsule | | | | |
|---|---|---|---|---|---|
| Ingredients | 4A | 4B | 4C | 4D | 4E |
| Compound (I) | 0.5 | 1.0 | 2.5 | 5.0 | 10.0 |
| Lactose monohydrate | 6.0 | 6.0 | 6.0 | 3.0 | 4.0 |
| (Respitose ML006) | | | | | |
| Lactose monohydrate | 13.5 | 13.0 | 11.5 | 12.0 | 6.0 |
| (Respitose SV010) | | | | | |
| Total in mg | 20.0 | 20.0 | 20.0 | 20.0 | 20.0 |

Manufacturing process of example 4 composition was similar to the above example 1

Stability studies was carried out for example 4A compositions filled-in a capsule were loaded in a HDPE with 1 gm silica and induction sealed.

TABLE 5A
Summary of Results (Accelerated) 40° C. ± 2° C. & 75% ± 5% RH for example 4A

| | | | 1 | 2 | 3 | 6 | 12 | 24 |
|---|---|---|---|---|---|---|---|---|
| Test | Specification | Initial | month | months | months | months | months | months |
| Assay | NLT | 98.8 | 102.5 | 101.2 | 102.4 | 100 | 100.4 | 102.2 |
| | 90% and | | | | | | | |
| | NMT | | | | | | | |
| | 110% | | | | | | | |
| Related | Impurity | 0.29 | 0.28 | NP | 0.29 | 0.28 | 0.28 | 0.28 |
| substances | A (NMT | | | | | | | |
| | 0.5%) | | | | | | | |
| | Individual | 0.05 | 0.07 | NP | 0.09 | 0.07 | 0.13 | 0.14 |
| | impurity | | | | | | | |
| | (NMT 1%) | | | | | | | |
| | Total | 0.45 | 0.45 | NP | 0.52 | 0.39 | 0.64 | 0.72 |
| | impurity | | | | | | | |
| | (NMT 2%) | | | | | | | |
| Fine | NLT 50 | 152.23 mcg | 167.24 mcg | 165.21 mcg | 157.59 mcg | 144.83 mcg | 159.87 mcg | 131.72 mcg |
| particle | mcg | | | | | | | |
| mass | | | | | | | | |
| Delivered- | Average | 105.3 | NP | NP | 91.8 | 93.9 | NP | 100.1 |
| dose | 85% to | | | | | | | |
| uniformity | 115% | | | | | | | |
| | Min. 75% | 96.5 | NP | NP | 83 | 85.4 | NP | 77.9 |
| | Max. 125% | 120.6 | NP | NP | 99.7 | 101.9 | NP | 119.2 |

TABLE 5B
Summary of Results (Long Term) 25° C. ± 2° C. & 60% ± 5% RH for example 4A

| | | | 1 | 2 | 3 | 6 | 12 | 24 |
|---|---|---|---|---|---|---|---|---|
| Test | Specification | Initial | month | months | months | months | months | months |
| Assay | NLT | 100.1 | 103 | 102.9 | 99.9 | 98.5 | 97 | 96.3 |
| | 90% and | | | | | | | |
| | NMT | | | | | | | |
| | 110% | | | | | | | |
| Related | Impurity | 0.28 | 0.29 | NP | 0.27 | 0.28 | 0.27 | 0.27 |
| substances | A (NMT | | | | | | | |
| | 0.5%) | | | | | | | |
| | Individual | 0.04 | 0.04 | NP | 0.08 | 0.08 | 0.08 | 0.07 |
| | impurity | | | | | | | |
| | (NMT 1%) | | | | | | | |
| | Total | 0.42 | 0.42 | NP | 0.49 | 0.41 | 0.56 | 0.69 |
| | impurity | | | | | | | |
| | (NMT 2%) | | | | | | | |
| Fine | NLT 50 | 386.4 | 394.76 | 389.05 | 395.43 | 349.27 | 381.87 | 352.62 |
| particle | mcg | | | | | | | |
| mass | | | | | | | | |
| Delivered- | Average | 100.2 | NP | NP | 89.7 | 99.0 | NP | 95.1 |
| dose | 85% to | | | | | | | |
| uniformity | 115% | | | | | | | |
| | Min. 75% | 91.8 | NP | NP | 76.6 | 87.1 | NP | 87.0 |
| | Max. 125% | 114.3 | NP | NP | NP | NP | NP | 5 |

Stability studies was carried out for example 4E compositions filled-in a capsule were loaded in a HDPE with 1 gm silica and induction sealed.

TABLE 6A
Summary of Results (Accelerated) 40° C. ± 2°
C. & 75% ± 5% RH for example 4E

| Test | Specification | Initial | 1 month | 2 months | 3 months | 6 months |
|---|---|---|---|---|---|---|
| Assay | NLT 90% | 102.4 | 98.2 | 98.1 | 98.0 | 97.1 |
| | and NMT | | | | | |
| | 110% | | | | | |
| Related | Impurity A | 0.28 | 0.26 | NP | 0.28 | 0.27 |
| substances | (NMT | | | | | |
| | 0.5%) | | | | | |
| | Individual | 0.04 | 0.05 | NP | 0.10 | 0.10 |
| | impurity | | | | | |
| | (NMT 1%) | | | | | |
| | Total | 0.41 | 0.37 | NP | 0.51 | 0.52 |
| | impurity | | | | | |
| | (NMT 2%) | | | | | |
| Fine | NLT 50 | 4795.77 | 4644.54 | 4422.41 | 4221.05 | 4228.31 |
| particle | mcg | | | | | |
| mass | | | | | | |
| Delivered- | Average | 92.9 | NP | NP | 84.6 | 99.4 |
| dose | 85% to | | | | | |
| uniformity | 115% | | | | | |
| | Min. 75% | 86.4 | NP | NP | 80.0 | 92.6 |
| | Max. 125% | 102.7 | NP | NP | 101.7 | 107.8 |

TABLE 6B
Summary of Results (Accelerated) 40° C. ± 2° C. & 75% ± 5% RH for example 4E

| | | | 1 | 2 | 3 | 6 | 12 | 24 |
|---|---|---|---|---|---|---|---|---|
| Test | Specification | Initial | month | months | months | months | months | months |
| Assay | NLT 90% | 102.4 | 101.3 | 99.6 | 99.1 | 101.8 | 100.7 | 98.6 |
| | and NMT | | | | | | | |
| | 110% | | | | | | | |
| Related | Impurity | 0.28 | 0.26 | NP | 0.28 | 0.25 | 0.26 | 0.25 |
| substances | A (NMT | | | | | | | |
| | 0.5%) | | | | | | | |
| | Individual | 0.04 | 0.05 | NP | 0.11 | 0.1 | 0.1 | 0.09 |
| | impurity | | | | | | | |
| | (NMT 1%) | | | | | | | |
| | Total | 0.41 | 0.36 | NP | 0.59 | 0.52 | 0.63 | 0.77 |
| | impurity | | | | | | | |
| | (NMT 2%) | | | | | | | |
| Fine | NLT 50 | 4795.77 | 4872.83 | 5093.99 | 4568.79 | 4557.01 | 4392.51 | 4333.24 |
| particle | mcg | | | | | | | |
| mass | | | | | | | | |
| Delivered- | Average | 92.9 | NP | NP | 87.2 | 97.7 | NP | 92.8 |
| dose | 85% to | | | | | | | |
| uniformity | 115% | | | | | | | |
| | Min. 75% | 86.4 | NP | NP | 77.8 | 88.2 | NP | 85.0 |
| | Max. 125% | 102.7 | NP | NP | NP | NP | NP | 3.01 |

* NP means not performed

### Example 5: Formulations for Variable Flow Rate

The variable flow rate of compositions of example 5A to 5F were studied. The different trial lots selected for a single strength (2.5 mg) containing compound (I) of different particle sizes (PSD) with d₉₀ in the range of 3.1 microns to 4.9 microns. The variable flow rate at 30 L/minute, 60 L/minute, and 90 L/minute were assessed in this study using aerodynamic particle size distribution by Anderson Cascade Impactor.

TABLE 7

| | Example | Example | Example | Example | Example | Example |
|---|---|---|---|---|---|---|
| Ingredients | 5A | 5B | 5C | 5D | 5E | 5F |
| Compound (I) | 2.50 | 2.50 | 2.50 | 2.50 | 2.50 | 2.50 |
| Particle size | 4.85 | 4.20 | 3.78 | 3.73 | 3.18 | 3.18 |
| distribution of | | | | | | |
| compound (I) | | | | | | |
| Lactose | 5.50 | 5.50 | 6.00 | 5.50 | 6.00 | 5.50 |
| Monohydrate | | | | | | |
| (Respitose | | | | | | |
| ML006) | | | | | | |
| Lactose | 12.00 | 12.00 | 11.50 | 12.00 | 11.50 | 12.00 |
| Monohydrate | | | | | | |
| (Respitose | | | | | | |
| SV010) | | | | | | |

TABLE 8

| | Fine Particle Fraction | | | MMAD | | |
|---|---|---|---|---|---|---|
| | 90 | 60 | 30 | 90 | 60 | 30 |
| | L/ | L/ | L/ | L/ | L/ | L/ |
| Composition | minute | minute | minute | minute | minute | minute |
| Example 5A | 32.92 | 33.19 | 23.93 | 3.60 | 3.78 | 4.72 |
| Example 5B | 53.12 | 48.45 | 28.31 | 2.69 | 2.94 | 4.07 |
| Example 5C | 44.26 | 44.82 | 24.96 | 3.07 | 3.06 | 4.22 |
| Example 5D | 53.29 | 48.26 | 28.19 | 2.66 | 2.87 | 3.99 |
| Example 5E | 57.67 | 44.80 | 19.58 | 2.25 | 2.64 | 3.97 |
| Example 5F | 56.63 | 48.12 | 20.90 | 2.34 | 2.59 | 3.94 |

Conclusion: The flow rate had direct impact on fine particle fraction (FPF) and depends on the particle size of the drug substance. It is observed that maximum FPF was achieved at flow rate 90 L/minute and minimum was achieved at flow rate of 30 L/minute. However, there is no substantial difference between 90 L/minute & 60 L/minute values.

### Example 6: Ex-Vivo Evaluation of Compound (I) for IL-17 Inhibition from Bronchoalveolar Lavage (BAL) Cells from COPD Patients

### Methodology:

The test compound was prepared by 10 mM stock of Compound (I) was prepared in 100% DMSO. The saline was prepared by 0.85 gm of NaCl weighted and dissolved in 100 mL distilled water and adjusted the pH 7.0 to pH 7.4. It was then filter sterilized.

Preparation of PBS:—Dissolved 8 g of NaCl (137 mM), 0.2 g of KCl (2.7 mM), 1.44 g of Na₂HPO₄ (4.3 mM), 0.25 g of KH₂PO₄ (1.4 mM) in 1 L of Distilled water. Adjust the pH to 7.4 and autoclave the same.

### Bronchoscopy:

Flexible fiber optic bronchoscopy was performed after valid consent and lignocaine nebulization. Patient was given 2 mg of IV midazolam and monitored under pulse oximetry and cardiac monitor. BAL fluid was collected with repeated aspirations of saline preferably from middle lobe.

### Isolation of BAL Cells:

BALF was passed through cell strainer to remove debris and mucus. BAL cells were isolated from BALf by centrifugation at 800 rpm for 10 min.

### Treatment of the Cells:

BAL cells were seeded at a density of 1×10⁵ cells-2×10⁵ cells/well in a 96 well plate coated with Anti-human CD3 antibody (10 μg/mL) and anti-human CD2 antibody (2 μg/mL). The plate was shaken gently and cells incubated for 5-10 minutes in an incubator with 37° C. and 5% CO₂ to acclimatize.
1. Cells were pre-incubated for 60 minutes at 37° C. with serial dilutions of Compound (I). This was followed by the addition of Anti-human CD28 antibody (2 μg/mL).
2. Cells were incubated for 48-72 h in the incubator at 37° C. with 5% CO₂.
3. The plates were spun at 1200 rpm at 4° C. for 12 minutes, supernatant collected and used for IL17 estimation by ELISA.

Calculations: IL17 cytokine concentration in the supernatant was determined using linear regression from the standard graph and subsequently multiplied with respective dilution factors. Fold response (FR) was calculated as pg/ml of hIL17 in sample (treated) wells over pg/ml of hIL17 in Basal wells.

Percent Inhibition for each set treated with test compound was calculated by using the formula Percent Inhibition={100−(pg/mL of test compound/pg/mL of induced×100)} and IC₅₀ values were calculated from concentration response curves by nonlinear regression analysis using GraphPad Prism software.

### Results:

BAL cells released measurable levels of IL-17 falling in dynamic range of ELISA kits. Compound (I) showed a concentration dependent decrease of pro inflammatory cytokine IL17 from stimulated BAL cells with an IC₅₀ of 142.7 nM.>70% inhibition of IL17 release was seen at an ICmax concentration of 1-3 microns.

TABLE 9
COPD-BAL cells

| Compound | IL-17A |
|---|---|
| (I) | (% Inhibition) |
| 0.1 nM | 4.97 |
| 1 nM | 2.39 |
| 3 nM | 10.95 |
| 10 nM | 13.94 |
| 30 nM | 18.71 |
| 100 nM | 41.41 |
| 300 nM | 66.79 |
| 1 μM | 71.12 |
| 3 μM | 76.68 |
| 6 μM | 78.79 |
| 10 μM | 81.10 |
| IC₅₀ (nM) | 142.71 |

### Example 6: Ex-Vivo Evaluation of Compound (I) for IL-17 Inhibition from Peripheral Blood from COPD Patients

### Treatment of the Human Whole Blood:

1. ˜8 mL whole blood was collected per patient.
2. Human whole blood was diluted 1:1 with 0.85% NaCl.
3. Diluted blood was loaded on a 96 well plate. Blood was pre-incubated for 60 minutes at 37° C. with serial dilutions of Compound (I).
4. It was then transferred to 96 well plate coated with Anti-human CD3 antibody (final concentration—10 μg/mL). This was followed by the addition of Anti-human CD28 antibody (2 μg/mL).
5. Cells were incubated for 48 h in the incubator at 37° C. with 5% CO₂. The plates were spun at 1200 rpm at 4° C. for 12 minutes, supernatant collected and used for IL17 estimation by ELISA.

Calculations: IL17 cytokine concentration in the supernatant was determined using linear regression from the standard graph and subsequently multiplied with respective dilution factors. Fold response (FR) was calculated as pg/ml of hIL17 in sample (treated) wells over pg/ml of hIL17 in Basal wells.

Percent Inhibition for each set treated with test compound was calculated by using the formula

Percent Inhibition={100−(pg/mL of test compound/pg/mL of induced×100)}

IC₅₀ values were calculated from concentration response curves by nonlinear regression analysis using GraphPad Prism software.

### Results:

Compound (I) potently inhibited the release of proinflammatory cytokines IL17 from stimulated whole blood with an IC50 of 281.9 nM. (FIG. 4 and table 3)

TABLE 10
Whole Blood

| Compound | IL17 A |
|---|---|
| (I) | (% Inhibition) |
| 0.1 nM | 0.27 |
| 1 nM | 5.57 |
| 3 nM | 14.91 |
| 10 nM | 15.08 |
| 30 nM | 27.60 |
| 100 nM | 36.81 |
| 300 nM | 43.38 |
| 1 uM | 56.89 |
| 3 μM | 64.99 |
| 6 μM | 69.25 |
| 10 μM | 76.15 |
| 20 μM | 78.80 |
| IC₅₀ (nM) | 281.9 |

### Example 8: Particle Size Distribution Studies

The particle size of compound (I) was tested for various batches and is presented in the tables below.

TABLE 11
Particle size data during initial development
(in μm)

| API Batch | PSD retested results | | |
|---|---|---|---|
| No. | d₁₀ | d₅₀ | d₉₀ |
| A | 1.222 | 2.064 | 3.664 |
| B | 0.106 | 1.376 | 3.278 |
| C | 1.128 | 2.010 | 3.783 |

TABLE 12
Particle size data during intermediate development
(in microns)

| API Batch | PSD retested results | | |
|---|---|---|---|
| No | d₁₀ | d₅₀ | d₉₀ |
| D | 0.110 | 1.510 | 3.638 |
| E | 1.376 | 2.591 | 5.060 |
| F | 1.244 | 2.383 | 4.752 |

The particle size distribution studies were performed using Malvern Mastersizer 2000S using wet dispersion method. A sample of about 25 gram to 50 gram of compound (I) was transferred in a beaker, and 2-3 drops of nonidet P-40 and 4-5 drops of water were added to the sample in beaker, and 25-30 ml of water was added and sonicated for 180 seconds with continuous stirring, and the dispersion/solution of compound (I) was added in sample handling unit of Hydro 20000S. The particle size measurement was done using Malvern Mastersizer 2000S.

1-62. (canceled)

63. An inhalable composition comprising 2-{4-[(2S)-1,1-difluoro-2-hydroxypropyl]phenyl}-N-{4-[3-(2-ethylphenyl)pyrazine-2-yl] phenyl}acetamide (compound (I)) or a pharmaceutically acceptable salt thereof.

64. The composition of claim 63, wherein the composition is an inhalable dry powder.

65. The composition of claim 64, comprising a therapeutically effective dosage amount of 2-{4-[(2S)-1,1-difluoro-2-hydroxypropyl]phenyl}-N-{4-[3-(2-ethylphenyl)pyrazine-2-yl]phenyl}acetamide (compound (I)) or a pharmaceutically acceptable salt thereof and one or more pharmaceutically acceptable excipients.

66. The composition of claim 65, wherein the therapeutically effective dosage amount of the compound (I) is in the range of from about 0.1 mg to about 20 mg.

67. The composition of claim 65, wherein the composition is filled in a capsule or a blister or a sachet dosage form suitable for a monodose or a unit dose dry powder inhaler.

68. The composition of claim 65, wherein the one or more pharmaceutically acceptable excipients are selected from lactose, glucose, maltose, erythritol, sorbitol, raffinose, xylitol, maltitol, inositol, fructose, sucrose, dextrose, mannitol, trehalose, cyclodextrin and mixtures thereof.

69. The composition of claim 68, wherein the one or more pharmaceutically acceptable excipients is one or more inhalable grades of lactose.

70. The composition of claim 64, wherein the compound (I) is micronized.

71. The composition of claim 70, wherein the d90 particle size of compound (I) or a pharmaceutically acceptable salt thereof is less than about 10 microns.

72. The composition of claim 69, wherein the composition has a compound (I) to lactose weight ratio between from about 1:1 to about 1:100.

73. The composition of the claim 72, wherein the compound (I) to lactose weight ratio is selected from 1:1, 1:2, 1:3, 1:4, 1:5. 1:6, 1:7, 1:8, 1:9, 1:10, 1:11, 1:12, 1:13, 1:14, 1:15, 1:16, 1:17, 1:18, 1:19, 1:20, 1:21, 1:22, 1:23, 1:24, 1:25, 1:26, 1:27, 1:28, 1:29, 1:30, 1:31, 1:32, 1:33, 1:34, 1:35, 1:36, 1:37, 1:38, 1:39, 1:40, 1:41, 1:42, 1:43, 1:44, 1:45, 1:46, 1:47, 1:48, 1:49, and 1:50.

74. The composition of the claim 69, wherein the lactose has a d90 particle size in the range of from about 1 micron to about 200 microns.

75. The composition of the claim 69, wherein the composition has particles with a fine particle fraction in the range of from about 1% to about 99%.

76. The composition of the claim 69, wherein the composition has a fine particle mass about 5 mcg or more.

77. The composition of the claim 72, wherein the mass median aerodynamic diameter of compound (I) in said composition is in the range of from about 1 micron to about 6 microns.

78. The composition of the claim 72, wherein compound (I) has an aerodynamic particle size in the range of from about 0.1 microns to about 10 microns.

79. A method of inhibiting, suppressing, or treating a pulmonary disease in a subject in need thereof comprising administration of a composition comprising a therapeutically effective dosage amount of 2-{4-[(2S)-1,1-difluoro-2-hydroxypropyl]phenyl}-N-{4-[3-(2-ethylphenyl)pyrazine-2-yl] phenyl}acetamide (compound (I)) or a pharmaceutically acceptable salt thereof to the subject; wherein the therapeutically effective dosage amount of compound (I) is in the range of from about 0.1 mg to about 20 mg.

80. The method of the claim 79, wherein the composition is administered via inhalation to the subject.

81. A process for preparing an inhalable dry powder composition of claim 65 comprising the steps of:
(i) blending compound (I) or a pharmaceutically acceptable salt thereof with a part or full quantity of one or more excipient(s) or co-milling of compound (I) with one or more excipient(s);
(ii) optionally blending the mixture obtained in step (i) with the remaining part of excipient(s);
(iii) optionally conditioning the blend from step (i) or (ii); and
(iv) filling the final blend of step (i) or step (ii) or step (iii) in a capsule or a blister.

82. The process of the claim 81, wherein the excipient in step (i) is lactose, and step (i) comprises simple blending for at least for 10 minutes to 1 hour with the at a mixing speed of about 20 to about 40±10 RPM.

83. The process of the claim 82, wherein the process comprises simple blending of compound (I) with lactose having a d90 particle size less than about 100 microns to form the first blend, and final blending the first blend with lactose having a d90 particle size more than about 100 microns.
